(19) United States
(12) Patent Application Publication
(10) Pub. No.: US 2021/0128549 A1
(43) Pub. Date: May 6, 2021

(54) OXYCODONE AND METHYLNALTREXONE MULTIPARTICULATES AND SUSPENSIONS CONTAINING THEM

(71) Applicant: CELISTA PHARMACEUTICALS LLC, Edina, MN (US)

(72) Inventors: Yogesh DANDIKER, Edina, MN (US); Maulik Kiritkumar PANCHAL, Maple Grove, MN (US); Xiao YU, Maple Grove, MN (US)

(73) Assignee: CELISTA PHARMACEUTICALS LLC, Edina, MN (US)

(21) Appl. No.: 16/976,378

(22) PCT Filed: Feb. 28, 2019
(86) PCT No.: PCT/US19/20010

§ 371 Date: Aug. 27, 2020

Related U.S. Application Data
(60) Provisional application No. 62/636,680, filed on Feb. 28, 2018.

(51) Int. Cl. A61K 31/485; A61K 9/50; A61K 9/00; A61K 47/34; A61K 47/38; A61K 47/12; A61K 47/10; A61K 47/36; A61K 47/32; A61K 47/14; A61K 47/22; A61K 47/26; A61K 9/20; A61K 9/28; A61K 9/16

(52) U.S. Cl. CPC A61K 31/485; A61K 9/5078; A61K 9/0053; A61K 47/34; A61K 47/38; A61K 47/12; A61K 47/10; A61K 47/36; A61K 47/32; A61K 47/14; A61K 47/22; A61K 47/26; A61K 9/2031; A61K 9/2054; A61K 9/2013; A61K 9/2846; A61K 9/284; A61K 9/282; A61K 9/1694; A61K 9/5084

(57) ABSTRACT

The invention relates to a composition that can be administered orally, comprising (a) opioid multi-particulates comprising an opioid active agent, and an abuse resistant release controlling agent; and (b) opioid antagonist multi-particulates comprising an opioid antagonist, and a release controlling agent and/or a release delaying agent. It also relates to a liquid suspension that can be administered orally, comprising (a) opioid multi-particulates comprising an opioid active agent, and an abuse resistant release controlling agent; and/or (b) opioid antagonist multi-particulates comprising an opioid antagonist, and a release controlling agent and/or a release delaying agent; and wherein the multi-particulates are suspended in a liquid comprising a viscosity modifier and a flavoring agent. The invention also relates to kits containing these multi-particulates, and methods of making and using them.

### BACKGROUND

Opioids are effective analgesics frequently used to treat severe chronic pain arising in a variety of diseases and conditions, including post-operative pain and advanced cancer. However, the clinical efficacy of opioids is burdened by adverse reactions that can cause early discontinuation of administration, under-dosing, inadequate analgesia, and result in reduced quality of life. One of the adverse effects of taking opioids, such as oxycodone, morphine, hydromorphone, etc., is reduced bowel motility which can cause severe constipation.

Opioids relieve pain by activating opioid receptors in the central nervous system. However, opioids, also interact with receptors outside of the central nervous system, causing adverse effects including constipation, nausea, vomiting, and urinary retention. Constipation occurs in over 80% of the patients who receive long-term opioid treatment. Chronic constipation can result in hemorrhoids, rectal pain, and bowel obstruction, and extreme constipation can cause bowel rupture and death.

Traditional therapies to relieve constipation include bulking agents, stool softeners, stimulant laxatives, osmotic agents, and newer agents such as lubiprostone. The effects of such agents are nonspecific and can cause diarrhea, cramps and other adverse effects. In addition, these therapies are ineffective in relieving opioid-induced constipation in some patients.

Opioid-induced constipation is predominantly due to the activity of gastrointestinal (GI) μ-opioid receptors which are present throughout the GI tract. Selective inhibition of these receptors in patients receiving opioids can prevent or relieve constipation without interfering with the central nervous system mediated analgesic effects of opioids, and without causing withdrawal symptoms.

One means of avoiding opioid-induced constipation and other opioid-induced adverse effects in patients receiving opioids is to administer an opioid antagonist, such as methylnaltrexone or its salts, such as the bromide or sodium salt, to prevent the undesired μ-opioid receptor activation along the large intestine. Methylnaltrexone has been found to reduce the adverse effects of opioids without reducing their analgesic effect. This quaternary ammonium compound does not cross the blood-brain-barrier, and therefore, does not antagonize the central nervous system effects of opioids. Subcutaneous methylnaltrexone is approved for the acute treatment of opioid induced constipation. However, subcutaneous administration of methylnaltrexone produces only a short-term laxative effect on constipation rather than preventing it. There is evidence that, despite very low bioavailability, oral administration of methylnaltrexone can antagonize the opioid-induced delay of intestinal transit time.

Existing products containing methylnaltrexone are in the form of a unitary tablet or an injection, and are not optimal.

### SUMMARY OF INVENTION

The invention relates to a composition that can be administered orally, comprising: (a) opioid multi-particulates comprising (i) oxycodone or a pharmaceutically acceptable salt of oxycodone, and (ii) an abuse resistant release controlling agent; and/or (b) opioid antagonist multi-particulates comprising methylnaltrexone or a pharmaceutically acceptable salt of methylnaltrexone; wherein (i) the opioid antagonist multi-particulates are encapsulated with a release delaying agent, and/or (ii) the opioid antagonist multi-particulates comprise a release controlling agent; wherein the multi-particulates are suspended in a liquid comprising a viscosity modifier and a flavoring agent.

The invention also relates to a composition that can be administered orally, comprising: (a) opioid multi-particulates comprising (i) oxycodone or a pharmaceutically acceptable salt of oxycodone, and (ii) an abuse resistant release controlling agent; and (b) opioid antagonist multi-particulates comprising methylnaltrexone or a pharmaceutically acceptable salt of methylnaltrexone; wherein (i) the opioid antagonist multi-particulates are encapsulated with a release delaying agent; and/or (ii) the opioid antagonist multi-particulates comprise a release controlling agent.

The release delaying agent mentioned above is insoluble in solutions having a pH below a certain value, but is soluble in solutions with a pH above another value. For example, the release delaying agent is insoluble in solutions having a pH below about 5.0, 5.5, 6.0, 6.8 or 7.0 but soluble in solutions having a pH at or above the given pH.

The invention also relates to kits containing these multi-particulates, and methods of making and using the compositions.

The composition contains an opioid active agent to treat pain, and/or an opioid antagonist, to prevent or treat, in patients receiving opioids, opioid-related adverse effects, such as constipation, urinary retention, nausea, emesis, dysphoria, ileus, post-operative ileus, gastrointestinal dysfunction. Thus, in the methods of the invention the compositions are administered for these uses.

Many patients do not like receiving injections, particularly children. Furthermore, oral solid dosage forms, such as tablets and capsules, are not suitable for young children and infants who cannot swallow them, and are challenging for patients with a psychological aversion to swallowing tablets or a physical impairment to swallowing (dysphagia). Psychological aversion to swallowing tablets, which usually originates in childhood, is prevalent in all age groups, whereas dysphagia is much more common in older people.

In addition, solid oral dosage forms, such as tablets and capsules, do not provide easy dose titration according to a patient's weight or age.

The present invention has several advantages. Because it is multi-particulate, and can be administered orally as a liquid or sprinkled on food, it is much easier to administer than tablets, capsules and injections. The multi-particulates of the present invention also allow for dose-titration, such that a single formulation can be used for a wide weight and age range. These features are particularly important for pediatric and geriatric patients.

In addition, the the opioid multi-particulates of the present invention provide an abuse resistant formulation. Oxycodone is a schedule II opioid medication for moderate and severe pain. Although oxycodone is effective in managing pain, upon repeated administration or extended use, dependence and tolerance may develop. Individuals who are dependent on opioids may misuse or overdose it for non-therapeutic purpose. Individuals may crush, grind or extract extended release opioid products attempting to obtain the whole opioid dose for immediate absorption.

Moreover, the opioid multi-particulates of the present invention are designed to provide extended release of the opioid active agent, which allows for less frequent administration of the opioid. Furthermore, the opioid antagonist multi-particulates are designed to provide either extended release of the opioid antagonist throughout the gastrointestinal tract, and/or to provide delayed release at a specific location in the gastrointestinal tract to provide a local effect. Thus, the dosage forms of the invention are more convenient to administer and more flexible than existing opioid/opioid antagonist dosage forms, and may increase patient treatment compliance.

### BRIEF DESCRIPTION OF THE DRAWINGS

The foregoing and other features and aspects of the present technology can be better understood from the following description of embodiments and as illustrated in the accompanying drawings. The accompanying drawings, which are incorporated herein and form a part of the specification, further serve to illustrate the principles of the present technology.

FIG. 1 is an optical microscope image of pellets, and is representative of the appearance of a mixture of oxycodone and methylnaltrexone pellets.

FIG. 2 is a graph depicting an example of the expected in vitro release profile of opioid multi-particulates corresponding to Example 1.

FIG. 3 is a graph depicting an example of the expected in vitro release profile of opioid antagonist multi-particulates that are designed to release in a controlled manner throughout the gastrointestinal tract (GI tract).

FIG. 4 is a graph depicting an example of the expected in vitro release profile of opioid antagonist multi-particulates, corresponding to Example 2, that have a coating designed to prevent drug release in the stomach (under fasting conditions) and to start releasing the drug in the duodenum (pH 5.5).

FIG. 5 is a graph depicting an example of the expected in vitro release profiles of opioid multi-particulates and opioid antagonist multi-particulates, corresponding to Example 3. The curve plotted with triangular shaped points corresponds to oxycodone hydrochloride, and the curve plotted with circular shaped points corresponds to methylnaltrexone bromide.

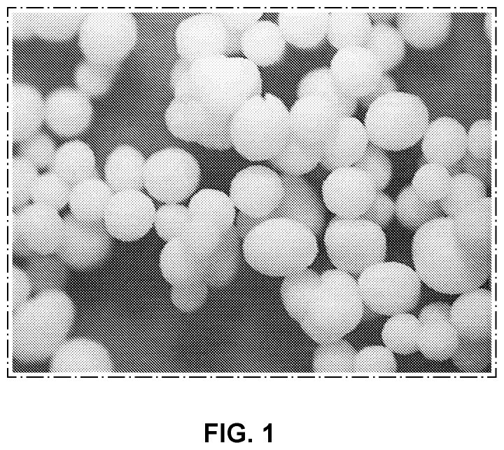

### DETAILED DESCRIPTION OF THE INVENTION

The invention provides an oral dosage from containing an opioid and/or an opioid antagonist in a multi-particulate form that is easy to swallow and allows for dose titration. The multi-particulates may be suspended in a liquid or sprinkled on soft food and ingested with the food. If the multi-particulates are large enough, e.g, if they are mini-tablets, they may be swallowed directly. The invention provides multi-particulate compositions, kits containing them, methods of making the compositions, and methods of treatment using these multi-particulates.

In one embodiment, the invention provides a kit comprising: (a) a container comprising opioid multi-particulates comprising (i) oxycodone or a pharmaceutically acceptable salt of oxycodone, and (ii) an abuse resistant release controlling agent; and/or (b) a container comprising opioid antagonist multi-particulates comprising methylnaltrexone or a pharmaceutically acceptable salt of methylnaltrexone, wherein the opioid antagonist multi-particulates (i) are encapsulated with a release delaying agent, and/or (ii) comprise a release controlling agent; and (c) a container comprising a suspending vehicle comprising a viscosity modifier and a flavoring agent.

In a preferred embodiment, the invention provides a kit comprising: (a) a container comprising opioid multi-particulates comprising oxycodone hydrochloride and an abuse resistant release controlling agent selected from the group consisting of: hydrogenated castor oil, carnuba wax, beeswax, glyceryl dibehenate, a polysaccharide, mixtures thereof, or a mixture of polyethylene oxide with an approximate molecular weight of from about 1,000,000 to about 2,000,000, and polyethylene oxide with an approximate molecular weight of from about 5,000,000 to about 7,000,000; and/or (b) a container comprising opioid antagonist multi-particulates comprising methylnaltrexone bromide; wherein (i) the opioid antagonist multi-particulates are encapsulated with a release delaying agent selected from the group consisting of: polyvinyl acetate phthalate, hypromellose phthalate, methacrylic acid and ethyl acrylate copolymer, methacrylic acid and methyl methacrylate copolymer (1:1), cellulose acetate phthalate, hypromellose acetate succinate, poly (methyl acrylate-co-methyl methacrylate-co-methacrylic acid) 7:3:1, methacrylic acid and methyl methacrylate copolymer (1:2), and mixtures thereof; and/or (ii) the opioid antagonist multi-particulates comprise a release controlling agent selected from the group consisting of: hypromellose, polyvinyl acetate, ethyl cellulose, beeswax, carnauba wax, hydrogenated castor oil, glyceryl behenate, triglycerides, and mixtures thereof; and (c) a container comprising a suspending vehicle comprising a viscosity modifier, a flavoring agent, a filler-sweetener, and a pH modifier.

In addition, the invention provides a kit comprising: (a) a container comprising opioid multi-particulates comprising (i) oxycodone or a pharmaceutically acceptable salt of oxycodone, and (ii) an abuse resistant release controlling agent; and (b) a container comprising opioid antagonist multi-particulates comprising methylnaltrexone or a pharmaceutically acceptable salt of methylnaltrexone, wherein the opioid antagonist multi-particulates (i) are encapsulated with a release delaying agent; and/or (ii) comprise a release controlling agent.

In these kits, the opioid multi-particulates and the opioid antagonist multi-particulates can be in the same container, or in separate containers, and the suspending vehicle is in a separate container, or can be in the same container as either of the types of multi-particulates. If the suspending vehicle is in the same container as one of the multi-particulates, the multi-particulates have a protective coating, discussed further below.

The suspending vehicle can be a powder intended to be dissolved in water, or can be a liquid solution or dispersion, wherein the viscosity modifier and flavoring agent are dissolved.

The term multi-particulate means a plurality of (more than one) particles that can flow separately, i.e., they are not compressed together into a unitary dosage form. Multi-particulates encompass pellets, granules, and minitablets. Pellets are usually denser and more spherical than granules, because different processes are used to make each. Pellets can be made, for example, using the pelletizing process described in Example 1, below, and granules can be made, for example, using the granulation process described in Example 1. In a preferred embodiment, the multi-particulates are pellets spherical in shape, with a smooth surface and a sphericity above 0.80. Smoothness may be evaluated by observing microscope images of the multi-particulates. The sphericity (S) is the ratio of the perimeter of the equivalent circle, P_{EQPC}, to the real perimeter, Pᵣₑₐₗ. The result is a value between 0 and 1. The smaller the value, the more irregular the shape of the particle. Sphericity is measured by analyzing the microscope images and calculating the ratio stated above.

The multi-particulates contain an active pharmaceutical ingredient and inactive pharmaceutical ingredients. Describing multi-particulates as comprising certain ingredients means that all the particles in the group contain the listed ingredients.

The size of both the opioid and opioid antagonist multi-particulates that are pellets or granules is between about 100 μm to about 800 μm. More preferably the size of the multiparticulates is about 200 μm to about 500 μm. Preferably the size of the multi-particulates is below 600 μm, below 500 μm, below 400 μm, or below 300 μm. For uncoated particulates, the size may be between about 200 μm to about 400 μm, and for coated particulates the size may be about 300 μm to about 500 μm. These size ranges are ideal for suspension. Multi-particulate size can be measured by microscope or sieve analysis, for example 40-60 mesh US standard sieves provide multiparticulates of the size range about 250 to about 425 um. Herein, we use “size” to refer to the diameter, either actual if the particulate is spherical, or the diameter of a circle of equal projection area (EQPC), if not.

In another embodiment, the multi-particulates are minitablets having a diameter of about 1.0 mm to about 3.0 mm, depending on the tooling selected. These tablets are easier to swallow than regular sized tablets. Minitablets can be easily administered, e.g., by sprinkling on soft food such as apple sauce. They can be filled into capsules, sachets or other containers. The number of minitablets administered will depend on the desired dose.

The suspending vehicle is a viscous liquid that can suspend the multi-particulates to form a uniform suspension. This vehicle may contain a filler-sweetener, a viscosity modifier, a pH modifier, a flavoring agent and/or a surfactant. In the kit, the vehicle can be in the form of a uniform dry blend, which can be manufactured by mixing all the ingredients in a blender or granulating the blend in a high shear granulator, and packaging the dry mixture or granules in, e.g., a bottle, vial, or sachet.

The two types of multi-particulates may be in the same container, or in separate containers. In addition, the multi-particulates may be in the same container as the suspending vehicle dry blend, or this suspending vehicle dry blend may be in a separate container. At the time of use, water is added to the bottle or vial, or the sachet is poured into a cup, water is added, the multi-particulates are added (if they were in a separate container/s), and the dose is reconstituted into a uniform suspension.

Alternatively, the vehicle is in the form of a liquid that is manufactured by mixing and dissolving or suspending all the ingredients in a mixer and stirring for a few hours until a uniform suspension or clear solution is formed. The opioid multi-particulates and opioid antagonist multi-particulates can be packaged separately from this liquid and mixed together with the suspending agent liquid at the time of use.

Alternatively, with a protective coating, the opioid multi-particulates and/or the opioid antagonist multi-particulates may be mixed together with the suspending vehicle and provided as a ready-to-use suspension.

When a liquid is present in the packaging, a preservative may be added to avoid the growth of microorganisms.

The term container includes, but is not limited to, glass or plastic vials or bottles, and sachets.

The kits can further comprise a graduated syringe, a graduated cap, or a graduated spoon.

The invention also provides a method of preparing a composition by adding the opioid multi-particulates and the opioid antagonist multi-particulates to the suspending vehicle solution and mixing. In addition, the invention provides a method of preparing a composition comprising adding the opioid multi-particulates to a suspending vehicle solution and mixing. Furthermore, the invention provides a method of preparing a composition comprising adding the opioid antagonist multi-particulates to a suspending vehicle solution and mixing.

The opioid multi-particulates can be contained in a container closed with an abuse resistant lock. In addition, or alternatively, the opioid multi-particulates are resistant to crushing.

The invention further provides a composition comprising: (a) opioid multi-particulates comprising (i) oxycodone or a pharmaceutically acceptable salt of oxycodone, and (ii) an abuse resistant release controlling agent; and/or (b) opioid antagonist multi-particulates comprising methylnaltrexone or a pharmaceutically acceptable salt of methylnaltrexone, wherein the opioid antagonist multi-particulates (i) are encapsulated with a release delaying agent; and/or (ii) comprise a release controlling agent; wherein the multi-particulates are suspended in a liquid comprising a viscosity modifier and a flavoring agent.

In addition, the invention provides a composition comprising: (a) opioid multi-particulates comprising oxycodone hydrochloride and an abuse resistant release controlling agent selected from the group consisting of: hydrogenated castor oil, carnauba wax, beeswax, glyceryl dibehenate, a polysaccharide, mixtures thereof, or a mixture of polyethylene oxide with an approximate molecular weight of from about 1,000,000 to about 2,000,000, and polyethylene oxide with an approximate molecular weight of from about 5,000,000 to about 7,000,000; and (b) opioid antagonist multi-particulates comprising methylnaltrexone bromide; wherein (i) the opioid antagonist multi-particulates are encapsulated with a release delaying agent selected from the group consisting of: polyvinyl acetate phthalate, hypromellose phthalate, methacrylic acid and ethyl acrylate copolymer, methyl methacrylate copolymer (1:1), cellulose acetate phthalate, hypromellose acetate succinate, poly (methyl acrylate-co-methyl methacrylate-co-methacrylic acid) 7:3:1, methacrylic acid and methyl methacrylate copolymer (1:2), and mixtures thereof; and/or (ii) the opioid antagonist multi-particulates comprise a release controlling agent selected from the group consisting of: hypromellose, polyvinyl acetate, ethyl cellulose, beeswax, carnauba wax, hydrogenated castor oil, glyceryl behenate, triglycerides, and mixtures thereof; wherein the opioid multi-particulates and the opioid antagonist multi-particulates are suspended in a liquid comprising a viscosity modifier, a flavoring agent, a filler-sweetener, and a pH modifier.

Moreover, the invention provides a composition comprising: (a) opioid multi-particulates comprising (i) oxycodone or a pharmaceutically acceptable salt of oxycodone, and (ii) an abuse resistant release controlling agent; and (b) opioid antagonist multi-particulates comprising methylnaltrexone or a pharmaceutically acceptable salt of methylnaltrexone, wherein the opioid antagonist multi-particulates (i) are encapsulated with a release delaying agent; and/or (ii) comprise a release controlling agent.

In some embodiments of the kits and compositions of the invention, the kits or compositions include immediate release opioid multi-particulates, in addition to opioid multi-particulates containing an abuse resistant controlling agent. The immediate release opioid multi-particulates may be formulated with typical excipients. For example, the immediate release opioid multi-particulate may be formulated with an inert core (such as microcrystalline cellulose) coated with a mixture of the opioid and a binder, and other suitable excipients.

The preferred opioid active agent is a pharmaceutically acceptable salt of oxycodone, and the preferred opioid antagonist is a pharmaceutically acceptable salt of methylnaltrexone. Pharmaceutically acceptable salts of oxycodone include the hydrochloride (HCl), bitartrate, tartrate, pectinate, terephthalate and phosphate salts. Oxycodone hydrochloride is preferred. Pharmaceutically acceptable salts of methylnaltrexone include the bromide (Br) and sodium salts. Methylnaltrexone bromide is preferred.

The opioid multi-particulates may comprise, in addition to the opioid, an abuse-resistant release controlling agent, a filler, a plasticizer, a processing-aid, a protective agent, and/or a lubricant. Some excipients may have more than one function, for example, some excipients may be both a plasticizer and a processing-aid.

The opioid antagonist multi-particulates may comprise, in addition to the opioid antagonist, a release controlling agent, and/or a release delaying agent, and may also comprise a pH modifier-stabilizer, a filler, a binder, a pore-former, a flow aid, an antioxidant, a plasticizer, a processing-aid. Some excipients may have more than one function. For example, some excipients may be able to act as both a plasticizer and processing-aid.

The suspending vehicle, in addition to a viscosity modifier and a flavoring agent, may comprise a filler-sweetener, a pH modifier, a glidant, a preservative, and/or a colorant.

As used herein, the terms “a” and “an” mean one or more.

An example of the abuse resistant release controlling agent is polyethylene oxide having an approximate molecular weight, based on rheological measurements, of at least 1,000,000. Preferably, the abuse resistant release controlling agent comprises a mixture of two different approximate molecular weights, e.g., a polyethylene oxide with an approximate molecular weight of from about 1 million (M) to about 2M, and a polyethylene oxide with an approximate molecular weight of from about 5M to about 7M. Most preferably, it is a mixture of polyethylene oxide with an approximate molecular weight of 2M and polyethylene oxide with an approximate molecular weight of 7M.

For the purpose of this invention, the approximate molecular weight is based on rheological measurements. For example, polyethylene oxide is considered to have an approximate molecular weight of 1M when a 2% (by wt) aqueous solution of the polyethylene oxide using a Brookfield viscometer Model RVF, spindle No. 1, at 10 rpm, at 25° C. shows a viscosity range of 400 to 800 mPa s (cP). Polyethylene oxide is considered to have an approximate molecular weight of 2M when a 2% (by wt) aqueous solution of the polyethylene oxide using a Brookfield viscometer Model RVF, spindle No. 3, at 10 rpm, at 25° C. shows a viscosity range of 2000 to 4000 mPa s (cP). Polyethylene oxide is considered to have an approximate molecular weight of 5M when a 1% (by wt) aqueous solution of the polyethylene oxide using a Brookfield viscometer Model RVF, spindle No. 2, at 2 rpm, at 25° C. shows a viscosity range of 5500 to 7500 mPa s (cP). Polyethylene oxide is considered to have an approximate molecular weight of 7M when a 1% (by wt) aqueous solution of the polyethylene oxide using a Brookfield viscometer Model RVF, spindle No. 2, at 2 rpm, at 25° C. shows a viscosity range of 7500 to 10,000 mPa s (cP).

When polyethylene oxide is used as a coating for the multi-particulates, it is preferred that the manufacturing process include the following steps and parameters. First the particulate containing the opioid is prepared, e.g., by coating an inert core such as microcrystalline cellulose with the opioid, or by granulating the opioid with suitable excipients such as fillers and binders, to form a “drug particulate”. A fine or super fine grade of polyethylene oxide should be used. Preferably, the average particle size of the polyethylene oxide is below 100 um, or more preferably below 50 um, 30 um, 20 um, or 10 um. In addition, the ratio between the average particle size of the polyethylene oxide and the average particles size of the drug particulate should be 1:10 or lower, preferably 1:15 or lower, 1:20 or lower, 1:25 or lower, or 1:30 or lower. In preferred embodiments, this ratio is between 1:10 and 1:30. In other embodiments, this ratio is between 1:10 and 1:25 or between 1:15 and 1:30. These ranges include the values at the endpoints of the range.

The polyethylene oxide is suspended in an anhydrous organic solvent, such as isopropyl alcohol, or absolute ethanol. A binder, such as ethyl cellulose (or other known binders, including those described below), should also be included in the organic solvent. The polyethylene oxide, binder, organic solvent mixture is then used to coat the drug particulate, e.g., by spraying.

Thus, one embodiment of the invention is a composition comprising:
(a) opioid multi-particulates comprising pellets comprising microcrystalline cellulose and oxycodone hydrochloride to form opioid pellets having an average particle size, wherein the opioid pellets are coated with a coating comprising polyethylene oxide having an average particle size and a binder, wherein the ratio of the average particle size of the polyethylene oxide to the average size of the opioid pellets is from 1:10 to 1:30; and
(b) opioid antagonist multi-particulates comprising methylnaltrexone bromide.

In other embodiments these multiparticulates are in the form of a kit, or are suspended in a liquid, which may contain other ingredients such as viscosity modifiers, as described below.

Other abuse resistant release controlling agents that may be used include non-polymeric materials such as waxes or fatty acids, e.g. hydrogenated castor oil, carnauba wax, beeswax, or glyceryl dibehenate. Such ingredients can be used to form hard wax matrix multi-particulates. In addition, the abuse resistant release controlling agent can be a polymer such as a polysaccharide, that can form a thick, viscous hydrogel matrix which may be crosslinked.

The release controlling agent is an agent that slows release of the active ingredient from the multi-particulates. The agent may form a matrix containing the active ingredient, and/or may be a coating around a particulate, such as a pellet, that contains the active ingredient. As the particulate moves through the gastrointestinal tract, the matrix gradually erodes, or the coating forms pores, thereby releasing the active ingredient. The release controlling agent may be, for example, hypromellose, polyvinyl acetate, ethyl cellulose, or other polymeric materials such as waxes, fatty acids, or lipids, such as beeswax, carnauba wax, hydrogenated castor oil, glyceryl behenate, or triglycerides. The release controlling agent may also be a mixture of two or more of these agents. Where the release controlling agent is in the form of a coating, the particulate covered with the coating contains the active ingredient mixed with one or more fillers and/or binders or other pharmaceutical excipients.

The release delaying agent is an agent that dissolves in aqueous solutions in a pH dependent way. The release delaying agent is soluble in solutions having a given pH or a pH higher than that pH, but insoluble in solutions having a pH lower than this value. Thus, coating multi-particulates with a release delaying agent prevents release of the active ingredient from the multi-particulates until the multi-particulates are exposed to an aqueous environment having a pH at which the release delaying agent is soluble.

For example, the release delaying agent may be one that does not dissolve in solutions having a pH below 5.0, but does dissolve in solutions having a pH at or above about 5.0, such as polyvinyl acetate phthalate (phthalavin enteric coating polymer, PVAP) (e.g., found in Opadry® Enteric, 91 series), or hypromellose phthalate (HPMCP) (e.g., HP-50). Alternatively, the release delaying agent may be an agent that does not dissolve in solutions having a pH below about 5.5, but does dissolve in solutions having a pH at or above about 5.5, such as methacrylic acid and ethyl acrylate copolymer (e.g., found in Eudragit® L30-D55, Eudragit® L 100-55, or Acryl-EZE®), hypromellose phthalate (HPMCP) (e.g., HP-55 or HP-55S), or hypromellose acetate succinate (e.g., AquaSolve, AQOAT® AS-LF or LG). Alternatively, the release delaying agent may be one that does not dissolve in solutions having a pH below about 6.0, but does dissolve in solutions having a pH at or above about 6.0, such as methyl methacrylate copolymer (1:1) (e.g., Eudragit® L 100, or found in Eudragit® L 12,5 or Opadry® Enteric, 94 series), cellulose acetate phthalate, or hypromellose acetate succinate (e.g., AQOAT® AS-MF or MG). Alternatively, the release delaying agent may be one that does not dissolve in solutions having a pH below about 6.8, but does dissolve in solutions having a pH at or above about 6.8, such as hypromellose acetate succinate (e.g., AQOAT® AS-HF or HG). Alternatively, the release delaying agent may be one that does not dissolve in solutions having a pH below about 7.0, but does dissolve in solutions having a pH at or above about 7.0, such as poly (methyl acrylate-co-methyl methacrylate-co-methacrylic acid) 7:3:1 (e.g., Eudragit® FS 30D), poly(methacylic acid-co-methyl methacrylate) 1:2 (e.g., Eudragit® S100 or Eudragit® S 12,5, or found in Opadry® Enteric, 95 series).

The filler may be, for example, microcrystalline cellulose, lactose, starch, dextrose, dibasic calcium phosphate dehydrate, mannitol, or a mixture of two or more of these agents.

The plasticizer may be, for example, polyethylene glycol, propylene glycol, triethyl citrate, or a mixture of two or more of these agents. When polyethylene glycol is used, an antioxidant, such as Vitamin E succinate, may also be included in the formulation, particularly where heat is used during the formulation process.

The protective agent may be, for example, amino methacrylate copolymer (Eudragit® E 100), which is soluble in gastric juice up to pH 5.0 and swellable and permeable above pH 5.0. The purpose of the protective agent is to form a coating around the multiparticulate to help prevent ingredients from leaching out of the particulates while they are in the suspending vehicle, before they are ingested.

The lubricant may be, for example, magnesium stearate, colloidal silicon dioxide, talc, sodium stearyl fumarate, silica, fumed silica, calcium stearate, carnauba wax, or a mixture of two or more of these agents.

The flow aid, or glidant may be, for example colloidal silicon dioxide, magnesium oxide, magnesium silicate, magnesium trisilicate, talc, calcium phosphate tribasic, or a mixture of two or more of these agents.

The pH modifier-stabilizer may be, for example, citric acid, citrate salts, tartaric acid, tartrate salts, succinic acid, succinate salts, acetic acid, acetate salts, fumaric acid, fumarate salts, adipic acid, malic acid, or a mixture of two or more of these agents.

The binder may be, for example, ethyl cellulose, hypromellose (e.g., Methocel K4M, E5 or E15), hydroxypropyl cellulose, carboxy methyl cellulose sodium, povidone (e.g., Povidone K30), or a mixture of two or more of these agents. Optionally, the binder may also act as a pore-former.

The flavoring agent may be, for example, bubble gum flavor, peppermint flavor, cherry flavor, grape flavor, and orange flavor. The flavoring agent may also be a mixture of two or more agents, if they are compatible.

The viscosity modifier preferably increases the viscosity of the solution to which it is added, and may be, for example, sodium carboxymethyl cellulose, guar gum, xanthan gum, hydroxypropyl cellulose, methylcellulose, or a mixture of two or more of these agents.

The filler-sweetener may be, for example, sucrose, xylitol, mannitol, maltitol, sorbitol, sugar, or a mixture of two or more of these agents.

The preservatives may be, for example, methyl paraben, sodium benzoate, benzoic acid, sorbic acid, potassium sorbate, propionic acid, or a mixture of two or more of these agents.

The colorant may be, for example, FD&C red, blue or green.

The multi-particulates may include additional pharmaceutical ingredients. The suspension vehicle may also include additional pharmaceutical ingredients, such as surfactants.

The opioid multi-particulates and opioid antagonist multi-particulates of the invention may be characterized by their dissolution profiles.

Preferably, the opioid multi-particulates with an abuse resistant release controlling agent release not more than 60% of the oxycodone by 2 hours, not more than 80% by 4 hours, and not more than 100% by 6 hours, as measured using USP dissolution apparatus 1, with 900 ml of 0.1N HCl solution at 37° C., and a basket rotation speed of 100 rpm.

Alternatively, USP dissolution apparatus 2 paddle with a speed of 50-75 rpm may be used to evaluate dissolution rate. It is preferred that USP apparatus 2 be used when evaluating drug release from multiparticulates with higher doses of the active ingredients, e.g., a dosage unit containing 450 mg, or higher, methylnaltrexone bromide.

Preferably, the opioid antagonist multi-particulates with a release controlling agent release not more than 60% of the methylnaltrexone by 2 hours, not more than 80% by 4 hours, and not more than 100% by 6 hours, as measured using USP dissolution apparatus 1, with 900 ml of 0.1N HCl solution at 37° C., and a basket rotation speed of 100 rpm.

In another embodiment, the opioid antagonist multi-particulates with a release controlling agent release not more than 60% of the methylnaltrexone by 2 hours, not more than 80% by 4 hours, and not more than 100% by 6 hours, as measured using USP dissolution apparatus 2, with 900 ml of 0.1N HCl solution at 37° C., and a paddle rotation speed of 50-75 rpm.

Preferably the delayed release (designed to release at pH 5.5 and above) opioid antagonist multi-particulates release not more than 40% of the methylnaltrexone by 2 hours, not more than 60% by 4 hours, and not more than 80% by 8 hours, as measured using USP dissolution apparatus 1, with 900 ml of 0.1N HCl solution at 37° C. for 1 hr, and then switching to phosphate buffer at pH 5.5, with a basket rotation speed of 100 rpm.

In another embodiment, the delayed release (designed to release at pH 5.5 and above) opioid antagonist multi-particulates release not more than 40% of the methylnaltrexone by 2 hours, not more than 60% by 4 hours, and not more than 80% by 8 hours, as measured using USP dissolution apparatus 2, with 900 ml of 0.1N HCl solution at 37° C. for 1 hr, and then switching to phosphate buffer at pH 5.5, with a paddle rotation speed of 50-75 rpm.

Preferably the delayed release (designed to release at pH 7.0 and above) opioid antagonist multi-particulates release not more than 20% of the methylnaltrexone by 4 hours, and not more than 60% by 8 hours, as measured using USP dissolution apparatus 1, with 900 ml of 0.1N HCl solution at 37° C. for 1 hr, then changing the media to phosphate buffer at pH 5.5 for 3 hrs, and then changing the media to phosphate buffer at pH 7.0, with a basket rotation speed of 100 rpm.

In another embodiment, the delayed release (designed to release at pH 7.0 and above) opioid antagonist multi-particulates release not more than 20% of the methylnaltrexone by 4 hours, and not more than 60% by 8 hours, as measured using USP dissolution apparatus 2, with 900 ml of 0.1N HCl solution at 37° C. for 1 hr, then changing the media to phosphate buffer at pH 5.5 for 3 hrs, and then changing the media to phosphate buffer at pH 7.0, with a paddle rotation speed of 50-75 rpm.

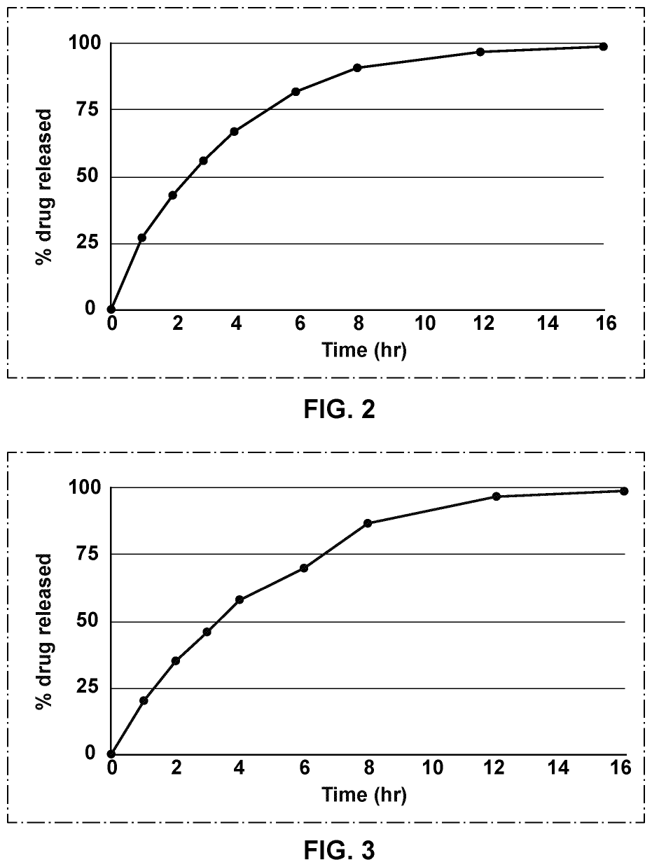

Some of the embodiments of the multi-particulates of this invention have the predicted in vitro release profiles depicted in one of FIGS. 2-5.

The USP apparatus 1 dissolution with 0.1 N HCl dissolution media is used to obtain the in vitro release profiles of FIG. 2 (oxycodone hydrochloride extended release multi-particulates) and FIG. 3 (methylnaltrexone bromide extended release multi-particulates). Each multi-particulate sample is placed in a basket in 900 mL 0.1 N HCl dissolution media at a temperature of 37° C., with a basket rotation speed of 100 rpm for a total of 16 hours.

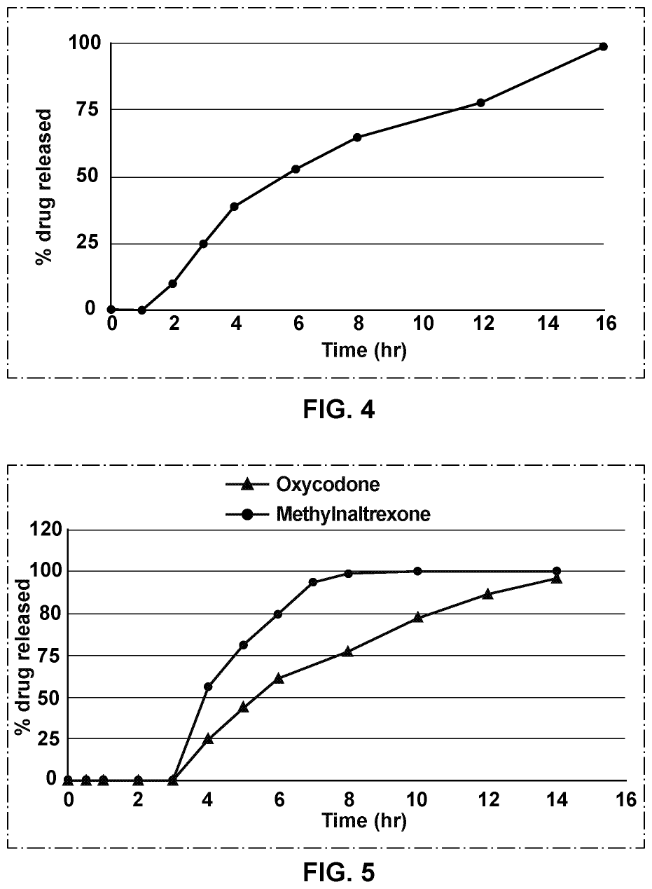

The in vitro release profile of FIG. 4 (methylnaltrexone bromide multi-particulates with delayed release coating designed to release at pH 5.5 and above) is obtained by subjecting the multi-particulates to a USP I (Basket) dissolution test at 100 rpm in 900 mL of 0.1 N HCl at 37° C. for 1 hr and then changing to phosphate buffer at pH 5.5.

The in vitro release profile from FIG. 5 (opioid multi-particulates and opioid antagonist multi-particulates, corresponding to Example 3) is obtained by subjecting the multi-particulates to a USP I (Basket) dissolution test at 100 rpm in 900 mL of 0.1 N HCl at 37° C. for 1 hr, then changing the media to phosphate buffer at pH 5.5 for 3 hrs, and then changing the media to phosphate buffer at pH 7.0.

The kits and compositions comprising opioid multi-particulates can be administered to treat pain. The kits and compositions comprising opioid antagonist multi-particulates can be administered to treat or prevent opioid-induced constipation, urinary retention, nausea, emesis, dysphoria, ileus, post-operative ileus or gastrointestinal dysfunction. Preferably, the kits and compositions comprising opioid antagonist multi-particulates can be administered to treat or prevent opioid-induced constipation. The kits and compositions comprising both types of multi-particulates can be administered both to treat pain, and to treat or prevent opioid-induced constipation, urinary retention, nausea, emesis, dysphoria, ileus, post-operative ileus or gastrointestinal dysfunction.

The compositions of the invention provide for extended release of the active ingredients, allowing for administration of a unit dose once per day.

The liquid oral suspensions of the invention are particularly useful for administration to a subject less than 8 years of age or greater than 60 years of age. However, the suspension is also a convenient and useful dosage form for all age groups.

The preferred dose of oxycodone hydrochloride in adults is about 0.05 to about 1.15 mg/kg bodyweight of the subject, and the preferred dose of methylnaltrexone bromide is about 0.5 to about 11.5 mg/kg bodyweight of the subject, more preferably about 5.7 mg/kg bodyweight of the subject.

The recommended dose for oxycodone in pediatric patients is 0.05-0.15 mg/kg body weight. The currently established highest safe dose for methylnaltrexone is 19.2 mg/kg body weight. (Yuan, V. S., et al., Clin. Pharmacol. Ther., April 1997; 61(4):467-75.)

The pediatric dose for methylnaltrexone injection is 0.15 mg/kg, and for oral methylnaltrexone is 6.4 mg/kg. Patients with severe renal impairment (creatinine clearance <30 mL/min) should receive half of the calculated methylnaltrexone dose to account for prolonged clearance.

In the kits, compositions and methods of the invention, the ratio of oxycodone hydrochloride dose to methylnaltrexone bromide dose is preferably from about 1:5 to about 1:15, most preferably about 1:10. Thus, for example, a unit dose for a dosage form containing both of these ingredients would preferably contain these ingredients in the dose combinations described in the following table:

TABLE 1

| Oxycodone | Methylnaltrexone | |
|---|---|---|
| Hydrochloride | Bromide Dose | |
| Dose (mg) | (mg) | Comments |
| 5 | 50 | For pediatric patients, 5 mg is the |
| | | maximum dose of oxycodone for |
| | | children who are opioid naïve with |
| | | moderate to severe pain. |
| | | Dose given based on weight at |
| | | 0.05-0.15 mg/kg. |
| 10 | 100 | For pediatric patients with severe |
| 20 | 200 | chronic pain who are opioid tolerant, |
| 30 | 300 | and 11 years or older, start dose |
| 40 | 400 | based on current opioid intake. |
| 60 | 600 | |
| 80 | 800 | Current established highest and safe |
| | | dose for methylnaltrexone is 19.2 |
| | | mg/kg. [Yuan, VS et al., Clin. |
| | | Pharmacol. Ther., April 1997; |
| | | 61(4): 467-75.] |

The compositions of the invention can be administered under either fed or fasted conditions.

As used herein, the term “about” means±10% of the value that this term modifies.

Several embodiments of the invention are described herein. In addition, the invention is described with reference to the following examples. This invention is not limited to the embodiments or examples described herein. Modifications and variations may suggest themselves and are intended to be within the scope of the appended claims.

### EXAMPLES

### Example 1: Extended Release Oxycodone-Extended Release Methylnaltrexone Oral Suspension

(a) General Description

The formulation described in Table 2, below, is designed to release both the opioid and opioid antagonist in a controlled manner throughout the gastrointestinal tract.

TABLE 2

| | | | mg/unit |
|---|---|---|---|
| | | Ingredient | or |
| Description | Ingredient | Function | mL/unit |
| Oxycodone | Oxycodone | Active ingredient | 5-80 |
| Extended | hydrochloride | | |
| release | Polyethylene | Abuse resistant; | 5-80 |
| pellets | oxide Mw I | Release controlling | |
| | | agent | |
| | Polyethylene | Abuse resistant | 5-150 |
| | oxide Mw II | release controlling | |
| | | agent | |
| | Microcrystalline | Filler | 100-600 |
| | cellulose | | |
| | Water | Solvent | Quantity |
| | | | sufficient |
| | | | (q.s.) |
| Methylnaltrexone | Methylnaltrexone | Active Ingredient | 50-800 |
| bromide extended | bromide | | |
| release pellets | Citric acid | pH modifier- | 1-30 |
| | | stabilizer | |
| | Microcrystalline | Filler | 100-500 |
| | cellulose | | |
| | Hypromellose | Binder | 10-100 |
| | Ethyl cellulose | Release controlling | 50-300 |
| | | agent | |
| | Isopropyl alcohol | Organic solvent | Quantity |
| | | | sufficient |
| | | | (q.s.) |
| | Water | Solvent | q.s. |
| Suspending | Mannitol | Sweetener; filler | 100-500 |
| vehicle | Xanthan gum | Viscosity modifier | 60-480 |
| | Cherry flavor | Flavoring Agent | 1-20 |
| | Citric acid | pH modifier | 10-110 |
| | Sodium benzoate | Preservative | 10-100 |
| | FD&C Red | Colorant | 1-10 |
| | Water | Solvent | 30-60 |

The extended release oxycodone pellets described in Table 2 can be prepared by a pellet formation (pelletizing) process. Oxycodone hydrochloride, polyethylene oxide of two different approximate molecular weights (Mw I is from 1M to 2M and Mw II is from 5 M to 7M, and can be a combination of any within the range), and microcrystalline cellulose, are mixed and wetted with water in high shear granulator. The wet blend is then fed into a rotary granulator. Water is sprayed on the blend as the rotor rotates at a fixed speed forming granules in spherical shape. As the process continues, granules are further densified into pellets. The wet pellets are then dried in a fluid bed dryer, and cured in the fluid bed at 60° C. for at least 4 hours.

The extended release methylnaltrexone pellets described in Table 2 can be prepared by a conventional extrusion spheronization process. Methylnaltrexone bromide, microcrystalline cellulose, and citric acid, are blended to form a uniform mixture in a high shear granulator. A binder solution containing hypromellose is sprayed onto the blend and the blend is granulated for 2-3 minutes. The wet mass is formed and then fed into an extruder, which forms extrudates at different sizes based on dome die size, e.g., 0.6 mm. Extrudates are poured into a spheronizer bowl; as the spheronizer bowl rotates, spherical shape wet pellets are formed. The pellets are dried in a fluid bed processor. The extended release coating, containing ethyl cellulose dissolved in isopropyl alcohol and water, is sprayed onto the pellets at a fixed coating level to obtain the desired controlled release profile.

The suspending vehicle can be in the form of a uniform dry blend which can be manufactured by mixing all the ingredients, except water, in a blender, or granulating the ingredients in a high shear granulator, and packaging into a bottle or sachet with the pellets. At the time of use water is added to the bottle, or the sachet and water are added to a cup, and the dose is reconstituted into a uniform suspension. Alternatively, the suspending vehicle can be in the form of a liquid that is manufactured by mixing and dissolving all the ingredients in a mixer, and stirring for a few hours until a uniform clear solution is formed.

(b) Extended Release Oxycodone-Extended Release Methylnaltrexone Oral Suspension

The formulation described in Table 3, below, is designed to release both the opioid and opioid antagonist in a controlled manner throughout the gastrointestinal tract. The compositions described in this table can be made using the process described above.

TABLE 3

| | | mg/unit or |
|---|---|---|
| Description | Ingredient | mL/unit |
| Oxycodone Extended | Oxycodone hydrochloride | 40 |
| release pellets | Polyethylene oxide Mw 2,000,000 | 40 |
| | Polyethylene oxide Mw 7,000,000 | 60 |
| | Microcrystalline cellulose | 260 |
| | Water | q.s. |
| Methylnaltrexone | Methylnaltrexone bromide | 400 |
| bromide extended | Citric acid | 24 |
| release pellets | Microcrystalline cellulose | 400 |
| | Hypromellose | 48 |
| | Ethyl cellulose | 200 |
| | Isopropyl alcohol | q.s. |
| | Water | q.s. |
| Suspending vehicle | Mannitol | 348 |
| | Xanthan gum | 90 |
| | Cherry flavor | 5 |
| | Sodium benzoate | 60 |
| | FD&C Red | 5 |
| | Citric acid | 90 |
| | Water | 30 |

| | |
|---|---|
| TOTAL (solids in mg) | 2100 |

### Example 2: Extended Release Oxycodone and Methylnaltrexone Oral Suspension

(a) General Description

The formulation described in Table 4, below, is designed not to start releasing the opioid antagonist until the dosage form is exposed to an environment with a pH 5.0 or higher. The target release location for the opioid antagonist is the duodenum. The compositions described in this table can be made using the process described above.

TABLE 4

| | | | mg/unit |
|---|---|---|---|
| | | Ingredient | or |
| Description | Ingredient | Function | mL/unit |
| Oxycodone | Oxycodone | Active ingredient | 5-80 |
| extended | hydrochloride | | |
| release | Polyethylene | Abuse resistant; | 5-80 |
| granules | oxide Mw I | Release controlling | |
| | | agent | |
| | Polyethylene | Abuse resistant; | 5-150 |
| | oxide Mw II | Release controlling | |
| | | agent | |
| | Microcrystalline | Filler | 100-600 |
| | cellulose | | |
| | Isopropanol alcohol | Solvent | q.s. |
| | Povidone K30 | Binder | 10-100 |
| Methylnaltrexone | Methylnaltrexone | Active ingredient | 50-800 |
| bromide delayed | bromide | | |
| release pellets | Citric acid | pH modifier- | 1-30 |
| | | stabilizer | |
| | Microcrystalline | Filler | 100-500 |
| | cellulose | | |
| | Hypromellose | Binder | 10-100 |
| | Polyvinyl acetate | Release | 50-300 |
| | | controlling agent | |
| | Polyvinyl acetate | Release delaying | 50-300 |
| | phthalate | agent | |
| | Triethyl citrate | Plasticizer | 10-60 |
| | Water | Solvent | q.s. |
| Suspending | Xylitol | Sweetener; filler | 100-500 |
| vehicle | Guar gum | Viscosity | 60-480 |
| | | modifier | |
| | FD&C green | Colorant | 1-10 |
| | Peppermint flavor | Flavoring agent | 1-20 |
| | Citric acid | PH modifier | 10-110 |

In addition to the pellet formation process described in Example 1 for the extended release oxycodone pellets, a top granulation process may be used to prepare extended release oxycodone granules that control the drug release for a period of time and are resistant to crush and extraction. A top granulation process can be described as follows: oxycodone hydrochloride, microcrystalline cellulose, and polyethylene oxide at two different approximate molecular weights, are blended to form a uniform mixture. Povidone K30 is dissolved in the isopropanol alcohol to form a binder solution. The mixture is fed into a top spray fluid bed processor, where Povidone K30 solution is sprayed onto the powder mixture. Airflow and temperature are controlled throughout the fluid bed until particle size of the granules grow into the desired range, e.g. 300-500 μm. The granules are dried and sieved to be within the desired range. The granules are then cured in an oven for at least 4 hours at 60° C.

The methylnaltrexone bromide pellets can be prepared by a pellet formation process. Methylnaltrexone bromide, citric acid, and microcrystalline cellulose are blended in a high shear granulator, and water is sprayed onto the blend to produce a damp mass. The mass is then fed into a rotary processor. Additional water is sprayed onto the material as the rotor rotates at certain speed and wet pellets are formed and collected until the desired particle size is obtained. The wet pellets are dried in a fluid bed dryer, followed by functional coating with a dispersion of polyvinyl acetate and hypromellose. The coated extended release pellets are further coated with the release delaying agent, polyvinyl acetate phthalate, that will not release any opioid antagonist in an aqueous solution unless the aqueous solution has a pH of 5.0 or higher, such as is found in the duodenum.

The liquid suspending vehicle can be manufactured by adding the water to a mixer, and then adding the xylitol, guar gum, FD&C green, peppermint flavor, and citric acid separately, and stirring for a few hours until a uniform clear solution is formed.

The oxycodone and methylnaltrexone pellets are packaged separately from the suspending vehicle and are mixed together in the suspending vehicle at the time of use.

(b) Extended Release Oxycodone and Methylnaltrexone Oral Suspension

The formulation described in Table 5, below, is designed not to start releasing the opioid antagonist until the dosage form is exposed to an environment with a pH 5.0 or higher. The target release location for the opioid antagonist is the duodenum. The compositions described in this table can be made using the process described above.

TABLE 5

| | | mg/unit or |
|---|---|---|
| Description | Ingredient | mL/unit |
| Oxycodone extended | Oxycodone hydrochloride | 20 |
| release pellets | Polyethylene oxide Mw 1,000,000 | 20 |
| | Polyethylene oxide Mw 7,000,000 | 40 |
| | Microcrystalline cellulose | 130 |
| | Povidone K30 | 15 |
| | Isopropanol alcohol | q.s. |
| Methylnaltrexone | Methylnaltrexone bromide | 200 |
| bromide delayed | Citric acid | 12 |
| extended release pellets | Microcrystalline cellulose | 200 |
| | Hypromellose | 24 |
| | Polyvinyl acetate | 100 |
| | Polyvinyl acetate phthalate | 120 |
| | Triethyl citrate | 24 |
| | Water | q.s. |
| Suspending vehicle | Xylitol | 318 |
| | Guar gum | 90 |
| | FD&C green | 2 |
| | Colloidal silicon dioxide | 5 |
| | Peppermint flavor | 5 |
| | Citric acid | 90 |

| | |
|---|---|
| TOTAL (solids in mg) | 1415 |

### Example 3: Extended Release Oxycodone and Methylnaltrexone Oral Suspension

(a) General Description

The formulation described in Table 6, below, is designed not to start releasing the opioid antagonist until the dosage form is exposed to an environment with a pH 7.0 or higher. The target release location for the opioid antagonist is the ileum and colon.

The compositions described in this table can be made using the process described below.

TABLE 6

| | | | mg/unit |
|---|---|---|---|
| Description | Ingredient | Ingredient Function | or mL/unit |
| Oxycodone extended | Oxycodone hydrochloride | Active ingredient | 5-80 |
| release granules | Polyethylene oxide Mw I | Abuse resistant; | 5-80 |
| | | Release controlling | |
| | | agent | |
| | Polyethylene oxide Mw II | Abuse resistant; | 5-150 |
| | | Release controlling | |
| | | agent | |
| | Microcrystalline cellulose | Filler | 5-300 |
| | Polyethylene glycol 20,000 | Plasticizer/Processing | 1-60 |
| | | aid | |
| | Vitamine E succinate | Antioxidant | 1-60 |
| | Eudragit ® E 12.5 | Protective agent | 4-150 |
| Methylnaltrexone | Methylnaltrexone bromide | Active ingredient | 50-800 |
| bromide delayed | Citric acid | pH modifier-stabilizer | 1-30 |
| release pellets | Microcrystalline cellulose | Filler | 10-500 |
| | Hypromellose | Release controlling | 50-800 |
| | | agent | |
| | Poly (methyl acrylate-co- | Release delaying agent | 50-300 |
| | methyl methacrylate-co- | | |
| | methacrylic acid) 7:3:1 | | |
| | (Eudragit ® FS 30D) | | |
| | Propylene glycol | Plasticizer | 3-180 |
| | Water | Solvent | q.s. |
| Suspending vehicle | Sucrose | Filler-sweetener | 100-500 |
| | Sodium carboxymethyl | Viscosity modifier | 60-480 |
| | cellulose | | |
| | Bubblegum flavor | Flavoring agent | 1-20 |
| | Sodium benzoate | Preservative | 10-100 |
| | FD&C red | Colorant | 1-10 |
| | Citric acid | pH modifier | 10-110 |
| | Water | Solvent | 30-60 |

Hot melt extrusion is a solvent-free process that can produce dense extended release multiparticulates. Oxycodone extended release pellets may be prepared by the hot melt extrusion process, which is described as follows: the polyethylene oxide at two approximate molecular weights, oxycodone hydrochloride, microcrystalline cellulose (optional), vitamin E succinate and polyethylene glycol are mixed in a blender to form a uniform blend. The blend is then fed by a feeder at a controlled speed into the hot melt extruder where the temperature of melting and mixing zones on the hot melt extruder is maintained above the melting point of the polyethylene oxide polymers (68° C.), but far below the melting point of the active drug. The mixture containing molten polyethylene oxide is well mixed, densified and finally extruded through a die, which can further be processed into spherical shape pellets. These pellets are cured in the fluid bed for a minimum of 4 hours at 60° C. These pellets are sprayed with Eudragit® E 12.5, to provide a protective layer designed to prevent drug from releasing in the suspension before it can be administered.

Methylnaltrexone delayed release pellets are prepared by an extrusion spheronization process, like that described in Example 1, followed by coating in a fluid bed processor. Methylnaltrexone bromide, microcrystalline cellulose, hypromellose, and citric acid, are blended to form a uniform mixture in a high shear granulator. Water is sprayed onto the blend and the material is granulated for 2-3 minutes. A wet mass is formed and then fed into an extruder, which forms extrudates. Extrudates are poured into a spheronizer bowl; as the spheronizer bowl rotates (typically at 200-450 rpm), spherical shape wet pellets are formed, which can be dried in a fluid bed processor to form extended release matrix pellets. The pellets are sieved after drying to obtain 300-500 um size. These pellets are sprayed with a mixture of poly (methyl acrylate-co-methyl methacrylate-co-methacrylic acid) 7:3:1 (Eudragit® FS 30D), and propylene glycol in water. This delayed release coating protects the pellets in the stomach and upper small intestine. This coating prevents the pellets from releasing the active ingredient until they reach the ileum and colon, because the coating will not dissolve in an aqueous solution unless the aqueous solution has a pH of 7.0 or higher, as is found in the ileum and colon.

The liquid suspending vehicle is manufactured by adding the water to a mixer, and then adding the sucrose, sodium carboxymethyl cellulose, bubblegum flavor, sodium benzoate, FD&C red, and citric acid separately, and stirring for a few hours until a uniform clear solution is formed.

The oxycodone and methylnaltrexone pellets are packaged separately from the suspending vehicle and are mixed together at the time of use.

(b) Extended Release Oxycodone and Methylnaltrexone Oral Suspension

The formulation described in Table 7, below, is designed not to start releasing the opioid antagonist until the dosage form is exposed to an environment with a pH 7.0 or higher. The target release location for the opioid antagonist is the ileum and colon.

The compositions described in this table can be made using the process described above.

TABLE 7

| | | | mg/unit or |
|---|---|---|---|
| Description | Ingredient | Ingredient Function | mL/unit |
| Oxycodone extended | Oxycodone hydrochloride | Active ingredient | 5 |
| release granules | Polyethylene oxide Mw | Abuse resistant; | 5 |
| | 2,000,000 | Release controlling | |
| | | agent | |
| | Polyethylene oxide Mw | Abuse resistant; | 15 |
| | 5,000,000 | Release controlling | |
| | | agent | |
| | Microcrystalline cellulose | Filler | 10 |
| | Vitamin E succinate | Antioxidant | 2 |
| | Polyethylene glycol 20,000 | Plasticizer/Processing | 2 |
| | | aid | |
| Methylnaltrexone | Methylnaltrexone bromide | Active ingredient | 50 |
| bromide delayed | Citric acid | pH modifier-stabilizer | 1 |
| release pellets | Microcrystalline cellulose | Filler | 50 |
| | Hypromellose | Release controlling | 25 |
| | | agent | |
| | Poly (methyl acrylate-co- | Release delaying agent | 20 |
| | methyl methacrylate-co- | | |
| | methacrylic acid) 7:3:1 | | |
| | (Eudragit ® FS 30D) | | |
| | Propylene glycol | Plasticizer | 7 |
| | Water | Solvent | q.s. |
| Suspending vehicle | Sucrose | Filler-sweetener | 105 |
| | Sodium benzoate | Preservative | 60 |
| | FD&C red | Colorant | 3 |
| | Sodium carboxymethyl | Viscosity modifier | 90 |
| | cellulose | | |
| | Bubblegum flavor | Flavoring agent | 3 |
| | Citric acid | pH modifier | 90 |
| | Water | Solvent | 30 |

| | |
|---|---|
| TOTAL (solids in mg) | 540 |

### Example 4: Extended Release Oxycodone Suspension

The formulation described in Table 8, below, is designed to release oxycodone in a controlled manner throughout the gastrointestinal tract.

TABLE 8

| | | Ingredient | mg/unit |
|---|---|---|---|
| Description | Ingredient | Function | or mL/unit |
| Oxycodone extended | Oxycodone hydrochloride | Active ingredient | 10 |
| release pellets | Polyethylene oxide Mw | Abuse resistant; | 10 |
| | 1,000,000 | Release controlling | |
| | | agent | |
| | Polyethylene oxide Mw | Abuse resistant; | 17 |
| | 7,000,000 | Release controlling | |
| | | agent | |
| | Microcrystalline cellulose | Filler | 250 |
| Suspending vehicle | Maltitol | Filler-sweetener | 178 |
| | Hydroxypropyl cellulose | Viscosity modifier | 60 |
| | Peppermint flavor | Flavoring agent | 3 |
| | Sodium benzoate | Preservative | 50 |
| | FD&C green | Colorant | 2 |
| | Citric acid | pH modifier | 20 |
| | Water | Solvent | 20 |

| | |
|---|---|
| TOTAL (solids in mg) | 600 |

In addition to the hot melt extrusion, pellet formation and top granulation processes, described above, extended release oxycodone pellets may be prepared by an extrusion-spheronization process. Oxycodone hydrochloride, microcrystalline cellulose, and the two polyethylene oxides of different molecular weights are blended in a high shear granulator and sprayed with a mixture of solvent and water (e.g. isopropanol alcohol and water) to obtain wet mass. The wet mass is fed into an extruder and forms extrudates at the desired size. These extrudates are then poured into a spheronizer bowl. As the spheronizer bowl rotates for a few minutes, wet spherical pellets are formed. These pellets are dried and cured in a fluid bed processor for at least 4 hours at 60° C. The suspending vehicle may be prepared as described above in Examples 1-3.

### Example 5: Extended Release Oxycodone—Methylnaltrexone Minitablets

The formulation described in Table 9, below, contains opioid and opioid antagonist minitablets. The opioid minitablets are designed to release oxycodone in a controlled manner throughout the gastrointestinal tract. The opioid antagonist minitablets are also designed to release methylnaltrexone in a controlled manner throughout the gastrointestinal tract, unless the optional coating containing a release delaying agent is included, in which case, these minitablets will begin releasing methylnaltrexone when the minitablets reach the location in the intestine where the pH is high enough to dissolve this coating.

TABLE 9

| | | Ingredient | |
|---|---|---|---|
| Description | Ingredient | Function | mg/unit |
| Oxycodone extended | Oxycodone hydrochloride | Active ingredient | 5-80 |
| release minitablets | Polyethylene oxide Mw I | Abuse resistant; | 5-80 |
| | | Release controlling | |
| | | agent | |
| | Polyethylene oxide Mw II | Abuse resistant; | 5-150 |
| | | Release controlling | |
| | | agent | |
| | Microcrystalline cellulose | Filler | 5-300 |
| | Magnesium stearate | Lubricant | 0.5-10 |
| Methylnaltrexone | Methylnaltrexone bromide | Active ingredient | 50-800 |
| bromide delayed | Microcrystalline cellulose | Filler | 10-500 |
| and extended | Hypromellose | Release controlling | 2-500 |
| release minitablets | | agent | |
| | Magnesium stearate | Lubricant | 0.5-10 |
| | OPTIONAL COATING: | Release delaying | 10-300 |
| | Poly (methyl acrylate-co- | agent | |
| | methyl methacrylate-co- | | |
| | methacrylic acid) 7:3:1 | | |
| | (Eudragit ® FS 30D) or | | |
| | Polyvinyl acetate, | | |
| | Polyvinyl acetate | | |
| | phthalate, and Triethyl | | |
| | citrate | | |
| | Water | Solvent | q.s. |

TABLE 10

| Description | Ingredient | mg/unit |
|---|---|---|
| Oxycodone extended | Oxycodone hydrochloride | 80 |
| release minitablets | Polyethylene oxide Mw I | 80 |
| | Polyethylene oxide Mw II | 150 |
| | Microcrystalline cellulose | 200 |
| | Magnesium stearate | 5 |
| Methylnaltrexone | Methylnaltrexone bromide | 300 |
| bromide delayed | Microcrystalline cellulose | 180 |
| and extended | Magnesium stearate | 5 |
| release minitablets | Hypromellose | 300 |
| | Poly (methyl acrylate-co- | 200 |
| | methyl methacrylate-co- | |
| | methacrylic acid) 7:3:1 | |
| | Water | q.s. |

| | |
|---|---|
| TOTAL (solids in mg) | 1500 |

Oxycodone extended release minitablets can be prepared by mixing the active ingredient, a filler, the polyethylene oxide polymer(s), and a lubricant, magnesium stearate, in a blender first to form a uniform blend. The blend is then fed into an automatic minitablet press and compressed into 1-2 mm size minitablets. The minitablets are cured in an oven at 60° C. for a minimum of 4 hours.

Similarly, methylnaltrexone bromide minitablets can be prepared by blending the active ingredient, microcrystalline cellulose, hypromellose, and magnesium stearate, to form a uniform mixture. The blend is fed into an automatic minitablet press and compressed into 1-2 mm size extended release methylnaltrexone bromide minitablets. Optionally, these minitablets are coated in a fluid bed with a delayed release coating, such as poly (methyl acrylate-co-methyl methacrylate-co-methacrylic acid) 7:3:1, which can release the drug at a target location, e.g. the small intestine (pH 5.5), or at the ileum and colon (pH 7.0). Both oxycodone and methylnaltrexone minitablets are then filled into sachets.

### Example 6: Extended Release Methylnaltrexone Suspension

The formulation described in Table 11, below, is designed to release the opioid antagonist throughout the gastrointestinal tract.

The methylnaltrexone extended release pellets are prepared by the extrusion spheronization process followed by coating in a fluid bed processor, described above in Example 1. The suspending vehicle may be prepared as described above in Examples 1-3.

TABLE 11

| | | | mg/unit |
|---|---|---|---|
| Description | Ingredient | Ingredient Function | or mL/unit |
| Methylnaltrexone | Methylnaltrexone | Active | 50-800 |
| bromide extended | bromide | | |
| release pellets | Citric acid | pH modifier | 1-30 |
| | Microcrystalline | Filler | 10-500 |
| | cellulose | | |
| | Hypromellose | Binder | 2-50 |
| | Ethyl cellulose | Release controlling | 50-300 |
| | | agent | |
| | Isopropyl alcohol | Solvent | q.s. |
| | Water | Solvent | q.s. |
| Suspending vehicle | Sorbitol | Filler-sweetener | 50-500 |
| | Methyl cellulose | Viscosity modifier | 60-480 |
| | Cherry flavor | Flavoring agent | 1-20 |
| | Sodium benzoate | Preservative | 10-100 |
| | FD&C red | Colorant | 1-10 |
| | Citric acid | pH modifier | 20-100 |
| | Sodium lauryl | Surfactant | 1-20 |
| | sulfate | | |
| | Water | Solvent | 10-60 |

TABLE 12

| | | mg/unit |
|---|---|---|
| Description | Ingredient | or mL/unit |
| Methylnaltrexone | Methylnaltrexone | 450 |
| bromide extended | bromide | |
| release pellets | Citric acid | 30 |
| | Microcrystalline | 330 |
| | cellulose | |
| | Hypromellose | 50 |
| | Ethyl cellulose | 200 |
| | Isopropyl alcohol | q.s. |
| | Water | q.s. |
| Suspending vehicle | Sorbitol | 300 |
| | Methyl cellulose | 90 |
| | Sodium lauryl | 2 |
| | sulfate | |
| | Sodium benzoate | 50 |
| | FD&C red | 4 |
| | Cherry flavor | 4 |
| | Citric acid | 90 |
| | Water | 30 |

| | |
|---|---|
| TOTAL (solids in mg) | 1600 |

### Example 7: Delayed Release (Release at pH 5.5) Methylnaltrexone Suspension

The formulation described in Table 13, below, is designed not to start releasing the opioid antagonist until the dosage form is exposed to an environment with a pH 5.5 or higher. The target release location for the opioid antagonist is the duodenum.

The methylnaltrexone pellets and the suspending vehicle can be prepared by the processes illustrated in Examples 1-3.

TABLE 13

| | | | mg/unit |
|---|---|---|---|
| Description | Ingredient | Ingredient Function | or mL/unit |
| Methylnaltrexone | Methylnaltrexone | Active | 50-800 |
| bromide delayed | bromide | | |
| release pellets | Citric acid | pH modifier | 1-30 |
| | Microcrystalline | Filler | 10-500 |
| | cellulose | | |
| | Hypromellose | Binder | 2-50 |
| | Ethyl cellulose | Release controlling | 50-300 |
| | | agent | |
| | Isopropyl alcohol | Solvent | q.s. |
| | Methacrylic acid | Release delaying | 50-300 |
| | and ethyl acrylate | agent | |
| | copolymer | | |
| | (Eudragit L30-D55) | | |
| | Water | Solvent | q.s. |
| Suspending vehicle | Sugar | Filler-sweetener | 50-500 |
| | Guar gum | Viscosity modifier | 60-480 |
| | Grape flavor | Flavoring agent | 1-20 |
| | Citric acid | pH modifier | 20-100 |
| | Sodium benzoate | Preservative | 10-100 |
| | FD&C red | Colorant | 1-10 |
| | FD&C blue | Colorant | 1-10 |
| | Water | Solvent | 10-60 |

### Example 8: Delayed Release Methylnaltrexone Suspension, Release at pH 7.0

The formulation described in Table 14, below, is designed not to start releasing the opioid antagonist until the dosage form is exposed to an environment with a pH 7.0 or higher. The target release location for the opioid antagonist is the ileum and colon.

The methylnaltrexone pellets and the suspending vehicle can be prepared by the processes illustrated in Examples 1-3.

TABLE 14

| | | | mg/unit |
|---|---|---|---|
| Description | Ingredient | Ingredient Function | or mL/unit |
| Methylnaltrexone | Methylnaltrexone | Active | 50-500 |
| bromide delayed | bromide | | |
| release pellets | Citric acid | pH modifier | 1-30 |
| | Microcrystalline cellulose | Filler | 10-500 |
| | Hypromellose | Binder | 2-50 |
| | Ethyl cellulose | Release controlling | 50-300 |
| | | agent | |
| | Isopropyl alcohol | Solvent | q.s. |
| | Poly (methyl acrylate-co- | Release delaying | 10-300 |
| | methyl methacrylate-co- | agent | |
| | methacrylic acid) 7:3:1 | | |
| | Water | Solvent | q.s. |
| Suspending vehicle | Sorbitol | Filler-sweetener | 50-500 |
| | Hydroxypropyl cellulose | Viscosity modifier | 60-480 |
| | Cherry flavor | Flavoring agent | 1-20 |
| | Sodium benzoate | Preservative | 10-100 |
| | FD&C red | Colorant | 1-10 |
| | Citric acid | pH modifier | 20-100 |
| | Water | Solvent | 10-60 |

### Example 9: Extended Release Oxycodone and Methylnaltrexone Oral Suspension

The formulation described in Table 15, below, is designed to release both the opioid and opioid antagonist in a controlled manner throughout the gastrointestinal tract.

TABLE 15

| | | | mg/unit |
|---|---|---|---|
| Description | Ingredient | Ingredient Function | or mL/unit |
| Oxycodone Extended | Microcrystalline cellulose | Inert core | 100-600 |
| release pellets | pellets | | |
| | Oxycodone hydrochloride | Active ingredient | 5-80 |
| | Polyethylene oxide Mw I | Abuse resistant; | 5-80 |
| | | Release controlling | |
| | | agent | |
| | Polyethylene oxide Mw II | Abuse resistant | 5-150 |
| | | release controlling | |
| | | agent | |
| | Hydroxypropyl cellulose | Binder | 5-50 |
| | Isopropanol alcohol | Solvent | Quantity |
| | | | sufficient |
| | | | (q.s.) |
| | Water | Solvent | q.s. |
| Methylnaltrexone | Methylnaltrexone bromide | Active Ingredient | 50-800 |
| bromide extended | Citric acid | pH modifier-stabilizer | 1-30 |
| release pellets | Microcrystalline cellulose | Filler | 100-500 |
| | Hypromellose | Binder | 10-100 |
| | Ethyl cellulose | Release controlling | 50-300 |
| | | agent | |
| | Isopropyl alcohol | Solvent | Quantity |
| | | | sufficient |
| | | | (q.s.) |
| | Water | Solvent | q.s. |
| Suspending vehicle | Mannitol | Sweetener; filler | 100-500 |
| | Xanthan gum | Viscosity modifier | 60-480 |
| | Cherry flavor | Flavoring Agent | 1-20 |
| | Citric acid | pH modifier | 10-110 |
| | Sodium benzoate | Preservative | 10-100 |
| | FD&C Red | Colorant | 1-10 |
| | Water | Solvent | 30-60 |

The extended release oxycodone pellets described in Table 15 can be prepared by a single-step fluid bed coating process. Oxycodone hydrochloride and fine particle grade polyethylene oxide of two different molecular weights (Mw I is from 1M to 2M and Mw II is from 5 M to 7M, and can be a combination of any molecular weights within the range) are blended and dispersed in the isopropanol alcohol where hydroxypropyl cellulose is dissolved. This dispersion is sprayed onto the microcrystalline pellets. The pellets are then dried and cured in the fluid bed at 60° C. for at least 4 hours.

The extended release methylnaltrexone pellets and suspending vehicle described in Table 15 can be prepared by the process described in Example 1 for extended release methylnaltrexone pellets.

### Example 10: Extended Release Oxycodone and Methylnaltrexone Oral Suspension

The formulation described in Table 16 below is designed to release both the opioid and opioid antagonist in a controlled manner throughout the gastrointestinal tract.

TABLE 16

| | | mg/unit |
|---|---|---|
| Description | Ingredient | or mL/unit |
| Oxycodone Extended | Microcrystalline cellulose | 500 |
| release pellets | pellets | |
| | Oxycodone hydrochloride | 50 |
| | Polyethylene oxide Mw I | 25 |
| | Polyethylene oxide Mw II | 75 |
| | Hydroxypropyl cellulose | 40 |
| | Isopropanol alcohol | q.s. |
| Methylnaltrexone | Methylnaltrexone bromide | 400 |
| bromide extended | Citric acid | 24 |
| release pellets | Microcrystalline cellulose | 400 |
| | Hypromellose | 48 |
| | Ethyl cellulose | 200 |
| | Water | q.s. |
| | Isopropyl alcohol | q.s. |
| Suspending vehicle | Mannitol | 358 |
| | Xanthan gum | 90 |
| | Orange flavor | 5 |
| | Sodium benzoate | 60 |
| | FD&C Orange 1 | 5 |
| | Citric acid | 90 |
| | Water | 30 |

| | |
|---|---|
| TOTAL (solids in mg) | 2400 |

The extended release oxycodone pellets described in Table 16 can be prepared by a dry powder layering process. Oxycodone hydrochloride is dispersed in isopropanol alcohol where hydroxypropyl cellulose is dissolved to form a spraying dispersion. Fine particle grade polyethylene oxide of two different approximate molecular weights (Mw I is from 1M to 2M and Mw II is from 5 M to 7M, and can be a combination of any molecular weights within the range) are mixed and sprinkled in powder form together with the drug-binder solution onto microcrystalline spheres. As the solvents evaporated during this process, a layer of the drug, binder and polyethylene oxide is formed. The pellets are then dried and cured in the fluid bed at 60° C. for 4 hours.

The extended release methylnaltrexone pellets and suspending vehicle described in Table 16 can be prepared by the processes described in Example 1.

### Example 11: Extended Release Oxycodone Oral Suspension

The formulation described in Table 17 below is designed to release the opioid in a controlled manner throughout the gastrointestinal tract.

TABLE 17

| | | | mg/unit |
|---|---|---|---|
| Description | Ingredient | Ingredient Function | or mL/unit |
| Oxycodone extended | Microcrystalline cellulose | Filler | 100-600 |
| release pellets | Oxycodone hydrochloride | Active | 5-80 |
| | Hypromellose | Binder | 5-50 |
| | Polyethylene oxide Mw I | Release controlling | 5-80 |
| | | agent/abuse | |
| | | deterrent agent | |
| | Polyethylene oxide Mw II | Release controlling | 5-150 |
| | | agent/abuse | |
| | | deterrent agent | |
| | Povidone K30 | Binder | 5-50 |
| | Isopropanol alcohol | Solvent | q.s. |
| | Water | Solvent | q.s. |
| Suspending vehicle | Mannitol | Sweetener; filler | 100-500 |
| | Xanthan gum | Viscosity modifier | 60-480 |
| | Lemon flavor | Flavoring Agent | 1-20 |
| | Citric acid | pH modifier | 10-110 |
| | Sodium benzoate | Preservative | 10-100 |
| | FD&C Yellow 5 | Colorant | 1-10 |
| | Water | Solvent | 30-60 |

The oxycodone hydrochloride core pellets described in Table 17 can be prepared by an extrusion spheronization process. Oxycodone hydrochloride and microcrystalline cellulose are blended to form a uniform mixture in a high shear granulator. A binder solution containing hypromellose is sprayed onto the blend and the blend is granulated for several minutes. The wet mass is formed and then fed into an extruder, which forms extrudates at different sizes based on dome die size. Extrudates are poured into a spheronizer bowl; as the spheronizer bowl rotates, spherical shape wet pellets are formed. The core pellets are dried in a fluid bed dryer. Polyethylene oxide of two different molecular weights are then powder layered onto the dried pellets at a fixed coating level to obtain the desired controlled release profile and abuse deterrent property.

The suspending vehicle described in Table 17 can be prepared by the process for making a suspending vehicle described in Example 1.

### Example 12: Extended Release Oxycodone-Immediate Release Oxycodone Oral Suspension

The formulation described in Table 18 below, is designed to release the opioid in a controlled manner throughout the gastrointestinal tract.

TABLE 18

| | | | mg/unit |
|---|---|---|---|
| Description | Ingredient | Ingredient Function | or mL/unit |
| Oxycodone extended | Microcrystalline cellulose | Inert core | 100-600 |
| release pellets | pellets | | |
| | Oxycodone hydrochloride | Active ingredient | 1-80 |
| | Povidone K30 | Binder | 5-50 |
| | Polyethylene oxide Mw I | Abuse resistant; | 5-80 |
| | | Release controlling | |
| | | agent | |
| | Polyethylene oxide Mw II | Abuse resistant | 5-150 |
| | | release controlling | |
| | | agent | |
| | Isopropanol alcohol | Solvent | Quantity |
| | | | sufficient |
| | | | (q.s.) |
| | Water | Solvent | q.s. |
| Oxycodone immediate | Microcrystalline cellulose | Inert core | 100-600 |
| release pellets | pellets | | |
| | Oxycodone hydrochloride | Active ingredient | 1-80 |
| | Povidone K30 | Binder | 5-50 |
| | Isopropanol alcohol | Solvent | q.s. |
| | Water | Solvent | q.s. |
| Suspending vehicle | Mannitol | Sweetener; filler | 100-500 |
| | Xanthan gum | Viscosity modifier | 60-480 |
| | Mint flavor | Flavoring Agent | 1-20 |
| | Citric acid | pH modifier | 10-110 |
| | Sodium benzoate | Preservative | 10-100 |
| | Titanium dioxide | Colorant | 1-10 |
| | Water | Solvent | 30-60 |

Table 18 provides an example of a formulation containing two different portions of oxycodone loaded pellets. First, core pellets are prepared by a drug layering process in the fluid bed with Povidone K30 as the binder. Then oxycodone loaded core pellets are split into two portions. One portion is optionally coated with the controlled release polymer ethyl cellulose and the binder hypromellose. The other portion is coated with fine particle grade polyethylene oxide of two different molecular weights, which are dispersed in a solution of povidone K30 in isopropanol alcohol and water, dried and cured in the fluid bed. These two portions of coated pellets are blended together at a pre-determined ratio to reach the desired release rate. These two portions of pellets cannot be visually differentiated from each other.

### Example 13: Extended Release Oxycodone Oral Suspension

TABLE 19

| | | | mg/unit |
|---|---|---|---|
| Description | Ingredient | Ingredient Function | or mL/unit |
| Oxycodone extended | Microcrystalline cellulose | Filler | 100-600 |
| release pellets | Oxycodone hydrochloride | Active | 5-80 |
| | Hypromellose | Binder | 5-50 |
| | Polyethylene oxide Mw I | Release controlling | 5-80 |
| | | agent/abuse deterrent | |
| | | agent | |
| | Polyethylene oxide Mw II | Release controlling | 5-150 |
| | | agent/abuse deterrent | |
| | | agent | |
| | Ethyl cellulose | Binder | 5-150 |
| | Isopropanol alcohol | Solvent | q.s. |
| Suspending vehicle | Sugar | Sweetener; filler | 100-500 |
| | Guar gum | Viscosity modifier | 60-480 |
| | Cherry flavor | Flavoring Agent | 1-20 |
| | Citric acid | pH modifier | 10-110 |
| | Sodium benzoate | Preservative | 10-100 |
| | FD&C Red | Colorant | 1-10 |
| | Water | Solvent | 30-60 |

The oxycodone hydrochloride core pellets described in Table 19 can be prepared by an extrusion spheronization process. Oxycodone hydrochloride and microcrystalline cellulose are blended to form a uniform mixture in a high shear granulator. A binder solution containing hypromellose is sprayed onto the blend which is granulated for several minutes forming wet mass. The wet mass is then fed into an extruder, which forms extrudates at different sizes based on dome die size: e.g. 0.6 mm. Extrudates are poured into a spheronizer bowl; as the spheronizer bowl rotates, spherical shape wet core pellets around 600 μm size are formed. The core pellets are then dried in a fluid bed dryer. Next, fine particle size polyethylene oxide of two different molecular weights (less than 100 μm) are dispersed in isopropanol alcohol containing ethyl cellulose as a binder and sprayed onto the drug loaded core pellets to achieve the desired controlled release profile and abuse deterrent property.

The suspending vehicle described in Table 19 can be prepared by the process for making a suspending vehicle described in Example 1.

### Example 14 Extended Release Oxycodone Oral Suspension

TABLE 20

| | | | mg/unit |
|---|---|---|---|
| Description | Ingredient | Ingredient Function | or mL/unit |
| Oxycodone extended | Microcrystalline cellulose | Pellets | 100-800 |
| release pellets | Oxycodone hydrochloride | Active | 5-80 |
| | Kollidone K30 | Binder | 5-50 |
| | Water | Solvent | q.s. |
| | Polyethylene oxide Mw I | Release controlling | 5-80 |
| | | agent/abuse deterrent | |
| | | agent | |
| | Polyethylene oxide Mw II | Release controlling | 5-150 |
| | | agent/abuse deterrent | |
| | | agent | |
| | Ethyl cellulose | Binder | 5-150 |
| | Isopropanol alcohol | Solvent | q.s. |
| Suspending vehicle | Mannitol | Sweetener; filler | 100-500 |
| | Xanthan gum | Viscosity modifier | 60-480 |
| | Lemon flavor | Flavoring Agent | 1-20 |
| | Citric acid | pH modifier | 10-110 |
| | Sodium benzoate | Preservative | 10-100 |
| | FD&C Yellow 5 | Colorant | 1-10 |
| | Water | Solvent | 30-60 |

The oxycodone hydrochloride pellets described in Table 20 can be prepared by a drug layering process followed by a coating process. Oxycodone hydrochloride and Kollidone K30 are first dissolved in water to form a uniform solution. This solution is sprayed onto microcrystalline cellulose pellets up to 600 μm size to form drug layered core pellets which are dried in a fluid bed dryer. Fine particle size polyethylene oxide of two different molecular weights (less than 100 μm, preferably less than 30 μm) are dispersed in isopropanol alcohol containing ethyl cellulose as a binder and sprayed onto the drug loaded core pellets to achieve the desired controlled release profile and abuse deterrent property.

Several embodiments are specifically illustrated and/or described herein. However, it will be appreciated that modifications and variations of the disclosed embodiments are envisioned, and within the scope of the claims.

1-114. (canceled)

115. A kit comprising:
(a) a container comprising opioid multi-particulates comprising (i) oxycodone or a pharmaceutically acceptable salt of oxycodone, and (ii) an abuse resistant release controlling agent; and
(b) a container comprising opioid antagonist multi-particulates comprising methylnaltrexone or a pharmaceutically acceptable salt of methylnaltrexone, wherein (i) the opioid antagonist multi-particulates are encapsulated with a release delaying agent or (ii) the opioid antagonist multi-particulates comprise a release controlling agent.

116. The kit of claim 115 further comprising:
(c) a container comprising a suspending vehicle comprising a viscosity modifier and a flavoring agent.

117. The kit of claim 115, wherein the kit comprises (i) opioid antagonist multi-particulates comprising methylnaltrexone or a pharmaceutically acceptable salt of methylnaltrexone, and a release controlling agent; and (ii) opioid antagonist multi-particulates comprising methylnaltrexone or a pharmaceutically acceptable salt of methylnaltrexone, encapsulated with a release delaying agent.

118. The kit of claim 116, wherein the opioid multi-particulates and the opioid antagonist multi-particulates are in the same container, and the suspending vehicle is in a separate container.

119. The kit of claim 116, wherein the suspending vehicle is a liquid, wherein the viscosity modifier and flavoring agent are dissolved, and wherein the composition further comprises a filler-sweetener and pH modifier.

120. The kit of claim 119, wherein the multi-particulates are spherical in shape, with a sphericity above 0.80, and have a diameter of about 300 to about 400 μm.

121. The kit of claim 116, wherein the container comprising opioid multi-particulates comprises an abuse resistant lock.

122. The kit of claim 115, wherein the opioid antagonist multi-particulates comprise a release delaying agent and the release delaying agent is selected from the group consisting of: a release delaying agent that is insoluble in solutions having a pH below about 5.5, but soluble in solutions having a pH at or above about 5.5; and a release delaying agent that is insoluble in solutions having a pH below about 7.0, but soluble in solutions having a pH at or above about 7.0.

123. The kit of claim 115, wherein the multi-particulates are minitablets.

124. A method of preparing a composition from the kit of claim 115 comprising adding the opioid multi-particulates and the opioid antagonist multi-particulates to a liquid suspending vehicle comprising a viscosity modifier and flavoring agent and mixing the opioid multi-particulates, the opioid antagonist multi-particulates and the liquid suspending vehicle.

125. A kit comprising:
(1) a container comprising multi-particulates selected from the group consisting of: (a) opioid multi-particulates comprising (i) oxycodone or a pharmaceutically acceptable salt of oxycodone, and (ii) an abuse resistant release controlling agent, and (b) opioid antagonist multi-particulates comprising methylnaltrexone or a pharmaceutically acceptable salt of methylnaltrexone, wherein (i) the opioid antagonist multi-particulates are encapsulated with a release delaying agent or (ii) the opioid antagonist multi-particulates comprise a release controlling agent; and
(2) a container comprising a suspending vehicle comprising a viscosity modifier and a flavoring agent.

126. The kit of claim 125, wherein the suspending vehicle is a liquid, wherein the viscosity modifier and flavoring agent are dissolved, and wherein the composition further comprises a filler-sweetener and pH modifier.

127. The kit of claim 126, wherein the multi-particulates are spherical in shape, with a sphericity above 0.80, and have a diameter of about 300 to about 400 μm.

128. The kit of claim 125, wherein the opioid multi-particulates are in a container closed with an abuse resistant lock.

129. The kit of claim 125, wherein the kit comprises (i) opioid antagonist multi-particulates comprising methylnaltrexone or a pharmaceutically acceptable salt of methylnaltrexone, and a release controlling agent; and (ii) opioid antagonist multi-particulates comprising methylnaltrexone or a pharmaceutically acceptable salt of methylnaltrexone, encapsulated with a release delaying agent.

130. The kit of claim 129, wherein the opioid antagonist multi-particulates comprise a release delaying agent and the release delaying agent is selected from the group consisting of: a release delaying agent that is insoluble in solutions having a pH below about 5.5, but soluble in solutions having a pH at or above about 5.5; and a release delaying agent that is insoluble in solutions having a pH below about 7.0, but soluble in solutions having a pH at or above about 7.0.

131. The kit of claim 125, wherein the multi-particulates are minitablets.

132. A kit comprising:
(a) a container comprising opioid multi-particulates comprising oxycodone hydrochloride and an abuse resistant release controlling agent selected from the group consisting of: hydrogenated castor oil, carnauba wax, beeswax, glyceryl dibehenate, a polysaccharide, a mixture of polyethylene oxide having an approximate molecular weight of from about 1,000,000 to about 2,000,000 with polyethylene oxide having an approximate molecular weight of from about 5,000,000 to about 7,000,000, and mixtures thereof; and
(b) a container comprising opioid antagonist multi-particulates comprising methylnaltrexone bromide; wherein (i) the opioid antagonist multi-particulates are encapsulated with a release delaying agent selected from the group consisting of: polyvinyl acetate phthalate, hypromellose phthalate, methacrylic acid and ethyl acrylate copolymer, methyl methacrylate copolymer (1:1), cellulose acetate phthalate, hypromellose acetate succinate, poly (methyl acrylate-co-methyl methacrylate-co-methacrylic acid) 7:3:1, methacrylic acid and methyl methacrylate copolymer (1:2), and mixtures thereof; or (ii) the opioid antagonist multi-particulates comprise a release controlling agent selected from the group consisting of: hypromellose, polyvinyl acetate, ethyl cellulose, beeswax, carnauba wax, hydrogenated castor oil, glyceryl behenate, triglycerides, and mixtures thereof; and
(c) a container comprising a suspending vehicle comprising a viscosity modifier, a flavoring agent, a filler-sweetener, and a pH modifier.

133. A composition comprising:
(a) opioid multi-particulates comprising (i) oxycodone or a pharmaceutically acceptable salt of oxycodone, and (ii) an abuse resistant release controlling agent; and
(b) opioid antagonist multi-particulates comprising methylnaltrexone or a pharmaceutically acceptable salt of methylnaltrexone, wherein (i) the opioid antagonist multi-particulates are encapsulated with a release delaying agent or (ii) the opioid antagonist multi-particulates comprise a release controlling agent; and
wherein the opioid multi-particulates and the opioid antagonist multi-particulates are suspended in a liquid comprising a viscosity modifier and a flavoring agent.

134. The composition of claim 133, wherein the opioid multi-particulates release not more than 60% of the oxycodone by 2 hours, not more than 80% by 4 hours, and not more than 100% by 6 hours, as measured using USP dissolution apparatus 1, with 900 ml of 0.1N HCl solution at 37° C., and a basket rotation speed of 100 rpm; and wherein the opioid antagonist multi-particulates release not more than 40% of the methylnaltrexone by 2 hours, not more than 60% by 4 hours, and not more than 80% by 8 hours, as measured using USP dissolution apparatus 1, with 900 ml of 0.1N HCl solution at 37° C. for 1 hr, and then switching to phosphate buffer at pH 5.5, with a basket rotation speed of 100 rpm.

135. The composition of claim 133, wherein the opioid multi-particulates release not more than 60% of the oxycodone by 2 hours, not more than 80% by 4 hours, and not more than 100% by 6 hours, as measured using USP dissolution apparatus 1, with 900 ml of 0.1N HCl solution at 37° C., and a basket rotation speed of 100 rpm; and wherein the opioid antagonist multi-particulates release not more than 20% methylnaltrexone by 4 hours, and not more than 60% by 8 hours, as measured using USP dissolution apparatus 1, with 900 ml of 0.1N HCl solution at 37° C. for 1 hr, then changing the media to phosphate buffer at pH 5.5 for 3 hrs, and then changing the media to phosphate buffer at pH 7.0, with a basket rotation speed of 100 rpm.

136. The composition of claim 133, wherein the opioid multi-particulates release not more than 60% of the oxycodone by 2 hours, not more than 80% by 4 hours, and not more than 100% by 6 hours, as measured using USP dissolution apparatus 1, with 900 ml of 0.1N HCl solution at 37° C., and a basket rotation speed of 100 rpm; and wherein the opioid antagonist multi-particulates release not more than 60% of the methylnaltrexone by 2 hours, not more than 80% by 4 hours, and not more than 100% by 6 hours, as measured using USP dissolution apparatus 1, with 900 ml of 0.1N HCl solution at 37° C., and a basket rotation speed of 100 rpm.

137. The composition of claim 133, wherein the multi-particulates have a diameter below 600 μm.

138. The composition of claim 133, wherein the multi-particulates are spherical in shape, with a sphericity above 0.80, and have a diameter of about 300 to about 400 μm.

139. The composition of claim 133, wherein the opioid multi-particulates are resistant to crushing.

140. The composition of claim 133, wherein the composition comprises (i) opioid antagonist multi-particulates encapsulated with a release delaying agent and (ii) opioid antagonist multi-particulates comprising a release controlling agent.

141. The composition of claim 140, wherein the opioid antagonist multi-particulates comprise a release delaying agent and the release delaying agent is is selected from the group consisting of: a release delaying agent that is insoluble in solutions having a pH below about 5.5, but soluble in solutions having a pH at or above about 5.5; and a release delaying agent that is insoluble in solutions having a pH below about 7.0, but soluble in solutions having a pH at or above about 7.0.

142. A composition comprising opioid antagonist multi-particulates comprising methylnaltrexone or a pharmaceutically acceptable salt of methylnaltrexone, wherein (i) the opioid antagonist multi-particulates are encapsulated with a release delaying agent or (ii) the opioid antagonist multi-particulates comprise a release controlling agent; and wherein the opioid antagonist multi-particulates are suspended in a liquid comprising a viscosity modifier and a flavoring agent.

143. The composition of claim 142, wherein the opioid antagonist multi-particulates release not more than 40% of the methylnaltrexone by 2 hours, not more than 60% by 4 hours, and not more than 80% by 8 hours, as measured using USP dissolution apparatus 1, with 900 ml of 0.1N HCl solution at 37° C. for 1 hr, and then switching to phosphate buffer at pH 5.5, with a basket rotation speed of 100 rpm.

144. The composition of claim 142, wherein the opioid antagonist multi-particulates release not more than 20% of the methylnaltrexone by 4 hours, and not more than 60% by 8 hours, as measured using USP dissolution apparatus 1, with 900 ml of 0.1N HCl solution at 37° C. for 1 hr, then changing the media to phosphate buffer at pH 5.5 for 3 hrs, and then changing the media to phosphate buffer at pH 7.0, with a basket rotation speed of 100 rpm.

145. The composition of claim 142, wherein the opioid antagonist multi-particulates release not more than 60% of the methylnaltrexone by 2 hours, not more than 80% by 4 hours, and not more than 100% by 6 hours, as measured using USP dissolution apparatus 1, with 900 ml of 0.1N HCl solution at 37° C., and a basket rotation speed of 100 rpm.

146. The composition of claim 142, wherein the composition comprises (i) opioid antagonist multi-particulates encapsulated with a release delaying agent and (ii) opioid antagonist multi-particulates comprising a release controlling agent.

147. The composition of claim 146, wherein the opioid antagonist multi-particulates comprise a release delaying agent and the release delaying agent is selected from the group consisting of: a release delaying agent that is insoluble in solutions having a pH below about 5.5, but soluble in solutions having a pH at or above about 5.5; and a release delaying agent that is insoluble in solutions having a pH below about 7.0, but soluble in solutions having a pH at or above about 7.0.

148. The composition of claim 147, wherein the release delaying agent is selected from the group consisting of: polyvinyl acetate phthalate, hypromellose phthalate, methacrylic acid and ethyl acrylate copolymer, methyl methacrylate copolymer (1:1), cellulose acetate phthalate, hypromellose acetate succinate, poly (methyl acrylate-co-methyl methacrylate-co-methacrylic acid) 7:3:1, methacrylic acid and methyl methacrylate copolymer (1:2), and mixtures thereof.

149. The composition of claim 142, wherein the opioid antagonist multi-particulates comprise methylnaltrexone bromide, and wherein the release controlling agent is selected from the group consisting of: hypromellose, polyvinyl acetate, ethyl cellulose, beeswax, carnauba wax, hydrogenated castor oil, glyceryl behenate, triglycerides, and mixtures thereof.

150. The composition of claim 142, wherein the multi-particulates have a diameter below 600 μm.

151. The composition of claim 142, wherein the multi-particulates are spherical in shape, with a sphericity above 0.80, and have a diameter of about 300 to about 400 μm.

152. A composition comprising opioid multi-particulates comprising (i) oxycodone or a pharmaceutically acceptable salt of oxycodone, and (ii) an abuse resistant release controlling agent; wherein the opioid multi-particulates are suspended in a liquid comprising a viscosity modifier and a flavoring agent.

153. The composition of claim 152, wherein the opioid multi-particulates release not more than 60% of the oxycodone by 2 hours, not more than 80% by 4 hours, and not more than 100% by 6 hours, as measured using USP dissolution apparatus 1, with 900 ml of 0.1N HCl solution at 37° C., and a basket rotation speed of 100 rpm.

154. The composition of claim 152, wherein the opioid multi-particulates comprise oxycodone hydrochloride, and wherein the abuse resistant release controlling agent is selected from the group consisting of: hydrogenated castor oil, carnauba wax, beeswax, glyceryl dibehenate, a polysaccharide, mixtures thereof, or a mixture of a mixture of polyethylene oxide having an approximate molecular weight of 2,000,000 and polyethylene oxide having an approximate molecular weight of 7,000,000.

155. The composition of claim 152, wherein the multi-particulates have a diameter below 600 μm.

156. The composition of claim 152, wherein the multi-particulates are spherical in shape, with a sphericity above 0.80, and have a diameter of about 300 to about 400 μm.

157. The composition of claim 152, wherein the opioid multi-particulates are resistant to crushing.

158. A composition comprising:
(a) opioid multi-particulates comprising oxycodone hydrochloride and an abuse resistant release controlling agent selected from the group consisting of: hydrogenated castor oil, carnauba wax, beeswax, glyceryl dibehenate, a polysaccharide, a mixture of polyethylene oxide having an approximate molecular weight of from about 1,000,000 to about 2,000,000 with polyethylene oxide having an approximate molecular weight of from about 5,000,000 to about 7,000,000, and mixtures thereof; and
(b) opioid antagonist multi-particulates comprising methylnaltrexone bromide; wherein (i) the opioid antagonist multi-particulates are encapsulated with a release delaying agent selected from the group consisting of: polyvinyl acetate phthalate, hypromellose phthalate, methacrylic acid and ethyl acrylate copolymer, methyl methacrylate copolymer (1:1), cellulose acetate phthalate, hypromellose acetate succinate, poly (methyl acrylate-co-methyl methacrylate-co-methacrylic acid) 7:3:1, methacrylic acid and methyl methacrylate copolymer (1:2), and mixtures thereof; or (ii) the opioid antagonist multi-particulates comprise a release controlling agent selected from the group consisting of: hypromellose, polyvinyl acetate, ethyl cellulose, beeswax, carnauba wax, hydrogenated castor oil, glyceryl behenate, triglycerides, and mixtures thereof; and
wherein the opioid multi-particulates and the opioid antagonist multi-particulates are suspended in a liquid comprising a viscosity modifier and a flavoring agent, a filler-sweetener, and a pH modifier.

159. The composition of claim 158, wherein the multi-particulates have a diameter below 600 μm.

160. The composition of claim 158, wherein (a) the opioid multi-particulates comprise oxycodone hydrochloride, polyethylene oxide Mw I, polyethylene oxide Mw II; microcrystalline cellulose pellets, and a binder; and (b) the opioid antagonist multi-particulates comprise methylnaltrexone bromide, microcrystalline cellulose, ethyl cellulose, citric acid, and a binder; and (c) the opioid multi-particulates and the opioid antagonist multi-particulates are suspended in a liquid comprising mannitol, xanthan gum, citric acid, and a flavoring agent.

161. The composition of claim 158, wherein (a) the opioid multi-particulates comprise oxycodone hydrochloride and microcrystalline cellulose, the opioid multi-particulates are opioid pellets have an average particle size, the opioid pellets are coated with a coating comprising polyethylene oxide having an average particle size and a binder, and wherein the ratio of the average particle size of the polyethylene oxide to the average size of the opioid pellets is from 1:10 to 1:30; and (b) the opioid antagonist multi-particulates comprise methylnaltrexone bromide.

162. A composition comprising (a) opioid multi-particulates comprising (i) oxycodone or a pharmaceutically acceptable salt of oxycodone, and (ii) an abuse resistant release controlling agent; and (b) opioid antagonist multi-particulates comprising methylnaltrexone or a pharmaceutically acceptable salt of methylnaltrexone, wherein (i) the opioid antagonist multi-particulates are encapsulated with a release delaying agent or (ii) the opioid antagonist multi-particulates comprise a release controlling agent.

163. The composition of claim 162, wherein the multi-particulates are mini-tablets.

164. The composition of claim 162, wherein the multi-particulates have a diameter below 600 μm.

165. The composition of claim 162, wherein the opioid multi-particulates comprise oxycodone hydrochloride and the abuse resistant abuse controlling agent is selected from the group consisting of: hydrogenated castor oil, carnauba wax, beeswax, glyceryl dibehenate, a polysaccharide, mixtures thereof, or a mixture of a mixture of polyethylene oxide having an approximate molecular weight of 2,000,000 and polyethylene oxide having an approximate molecular weight of 7,000,000; and wherein the opioid antagonist multi-particulates comprising methylnaltrexone bromide; and (i) the opioid antagonist multi-particulates are encapsulated with a release delaying agent selected from the group consisting of: polyvinyl acetate phthalate, hypromellose phthalate, methacrylic acid and ethyl acrylate copolymer, methyl methacrylate copolymer (1:1), cellulose acetate phthalate, hypromellose acetate succinate, poly (methyl acrylate-co-methyl methacrylate-co-methacrylic acid) 7:3:1, methacrylic acid and methyl methacrylate copolymer (1:2), and mixtures thereof; or (ii) the opioid antagonist multi-particulates comprise a release controlling agent selected from the group consisting of: hypromellose, polyvinyl acetate, ethyl cellulose, beeswax, carnauba wax, hydrogenated castor oil, glyceryl behenate, triglycerides, and mixtures thereof.

166. The composition of claim 165, wherein the opioid antagonist multi-particulates comprise a release delaying agent and the release delaying agent is selected from the group consisting of: a release delaying agent that is insoluble in solutions having a pH below about 5.5, but soluble in solutions having a pH at or above about 5.5; and a release delaying agent that is insoluble in solutions having a pH below about 7.0, but soluble in solutions having a pH at or above about 7.0.

167. A method of treatment comprising administering to a subject a unit dose of the composition of claim 133, wherein the composition is administered to treat pain and to treat or prevent a condition selected from the group consisting of: opioid-induced constipation, urinary retention, nausea, emesis, dysphoria, ileus, post-operative ileus and gastrointestinal dysfunction.

168. A method of treatment comprising administering to a subject a unit dose of the composition of claim 133, wherein the composition is administered to treat pain and to treat or prevent opioid-induced constipation.

169. The method of claim 168, wherein the unit dose is administered only once per day.

170. A method of treatment comprising administering to a subject a unit dose of the composition of claim 133, wherein the age of the subject is less than 8 years of age or greater than 60 years of age.

171. A method of treatment comprising administering to a subject a unit dose of the composition of claim 133, wherein the opioid active agent is oxycodone hydrochloride and the dose administered of the opioid active agent is about 0.05 to about 1.15 mg/kg bodyweight of the subject, and wherein the opioid antagonist is methylnaltrexone bromide and the dose administered of the opioid antagonist is about 0.05 to about 0.2 mg/kg bodyweight of the subject.

172. A method of treatment comprising administering to a subject a unit dose of the composition of claim 133, wherein the opioid active agent is oxycodone hydrochloride and the opioid antagonist is methylnaltrexone bromide, and wherein the ratio of the oxycodone hydrochloride to the methylnaltrexone bromide in the unit dose is about 1:10.

173. A method of treatment comprising administering to a subject a unit dose of the composition of claim 142, wherein the composition is administered to treat or prevent a condition selected from the group consisting of: opioid-induced constipation, urinary retention, nausea, emesis, dysphoria, ileus, post-operative ileus and gastrointestinal dysfunction.

174. A method of treatment comprising administering to a subject a unit dose of the composition of claim 142, wherein the composition is administered to treat or prevent opioid-induced constipation.

175. The method of claim 173, wherein the unit dose is administered only once per day.

176. A method of treatment comprising administering to a subject a unit dose of the composition of claim 142, wherein the age of the subject is less than 8 years of age or greater than 60 years of age.

177. A method of treatment comprising administering to a subject a unit dose of the composition of claim 142, wherein the opioid antagonist is methylnaltrexone bromide, and the dose administered of the opioid antagonist is about 0.05 to about 0.2 mg/kg bodyweight of the subject.

178. A method of treatment comprising administering to a subject a unit dose of the composition of claim 152, wherein the composition is administered to treat pain.

179. The method of claim 178, wherein the unit dose is administered only once per day.

180. A method of treatment comprising administering to a subject a unit dose of the composition of claim 152, wherein the age of the subject is less than 8 years of age or greater than 60 years of age.

181. A method of treatment comprising administering to a subject a unit dose of the composition of claim 152, wherein the opioid active agent is oxycodone hydrochloride and the dose administered of the opioid active agent is about 0.05 to about 1.15 mg/kg bodyweight of the subject, and wherein the opioid antagonist is methylnaltrexone bromide and the dose administered of the opioid antagonist is about 0.05 to about 0.2 mg/kg bodyweight of the subject.

182. A method of treatment comprising administering to a subject a unit dose of the composition of claim 152, wherein the opioid active agent is oxycodone hydrochloride and the dose administered of the opioid active agent is about 0.05 to about 1.15 mg/kg bodyweight of the subject.

183. A method of treatment comprising administering to a subject a unit dose of the composition of claim 158 wherein the composition is administered to treat pain and to treat or prevent a condition selected from the group consisting of: opioid-induced constipation, urinary retention, nausea, emesis, dysphoria, ileus, post-operative ileus and gastrointestinal dysfunction.

184. A method of treatment comprising administering to a subject a unit dose of the composition of claim 158, wherein the composition is administered to treat pain and to treat or prevent opioid-induced constipation.

185. The method of claim 183, wherein the unit dose is administered only once per day.

186. A method of treatment comprising administering to a subject a unit dose of the composition of claim 158, wherein the age of the subject is less than 8 years of age or greater than 60 years of age.

187. A method of treatment comprising administering to a subject a unit dose of the composition of claim 158, wherein the opioid active agent is oxycodone hydrochloride and the dose administered of the opioid active agent is about 0.05 to about 1.15 mg/kg bodyweight of the subject, and wherein the opioid antagonist is methylnaltrexone bromide and the dose administered of the opioid antagonist is about 0.05 to about 0.2 mg/kg bodyweight of the subject.

188. A method of treatment comprising administering to a subject a unit dose of the composition of claim 158, wherein the opioid active agent is oxycodone hydrochloride and the opioid antagonist is methylnaltrexone bromide, and wherein the ratio of the oxycodone hydrochloride to the methylnaltrexone bromide in the unit dose is about 1:10.

189. A method of treatment comprising administering to a subject a unit dose of the composition of claim 162, wherein the composition is administered to treat pain and to treat or prevent a condition selected from the group consisting of: opioid-induced constipation, urinary retention, nausea, emesis, dysphoria, ileus, post-operative ileus and gastrointestinal dysfunction.

190. A method of treatment comprising administering to a subject a unit dose of the composition of claim 162, wherein the composition is administered to treat pain and to treat or prevent opioid-induced constipation.

191. The method of claim 189, wherein the unit dose is administered only once per day.

192. A method of treatment comprising administering to a subject a unit dose of the composition of claim 162, wherein the age of the subject is less than 8 years of age or greater than 60 years of age.

193. A method of treatment comprising administering to a subject a unit dose of the composition of claim 162, wherein the opioid active agent is oxycodone hydrochloride and the dose administered of the opioid active agent is about 0.05 to about 1.15 mg/kg bodyweight of the subject, and wherein the opioid antagonist is methylnaltrexone bromide and the dose administered of the opioid antagonist is about 0.05 to about 0.2 mg/kg bodyweight of the subject.

194. A method of treatment comprising administering to a subject a unit dose of the composition of claim 162, wherein the opioid active agent is oxycodone hydrochloride and the opioid antagonist is methylnaltrexone bromide, and wherein the ratio of the oxycodone hydrochloride to the methylnaltrexone bromide in the unit dose is about 1:10.
